SEQUENCE LISTING (155 sequences)

SEQ ID NO: 1 - DNA, 498 bp, Homo sapiens
  CDS (1)..(498)
gccccaccacgcttcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 2 - protein, 166 aa, Homo sapiens
APPRFICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTGEACRTGDR

SEQ ID NO: 3 - protein, 166 aa, Artificial sequence
  Erythropoetin variant
APPRFICDSRVLERYLLEAKEAENFTSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTEEACRTGDR

SEQ ID NO: 4 - protein, 166 aa, Artificial sequence
  Erythropoetin variant
APPRFICDSRVLERYLLEAKEAENIATGCAEHCSLNENITVPGTKVNFYAWKRMEVGQQAAEVWQGLALLSEAVRRGQALLANSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 5 - protein, 166 aa, Artificial sequence
  Erythropoetin variant
APPRFICDSRVLERYLLEAKEAEDITAGCAEHCSLNENIAVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 6 - protein, 166 aa, Artificial sequence
  Erythropoetin variant
APPRFTCGSRVLERYLLEAREAENITTGCAEHCSLNENITVPDTKVNFYAWRRMEVGQQAVEVRQGLALLSEAFLRGQALLVNSSQPWEPLQLHVDKAVSGLRSLTALFRALGAQEEAISPPDAASAAPLRTNTADTFRKLFRVYSNFLRGKLKLYIGEACRTGDR

SEQ ID NO: 7 - protein, 166 aa, Artificial sequence
  Erythropoetin variant
APPRFICDSRVLERYLLEAKEAENFTTGVAEHASLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 8 - protein, 166 aa, Artificial sequence
  Erythropoetin variant
APPRFICDSRVLERYLLEAKEAENIAAGCAEHCSLNENITVPDTRVDFNAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 9 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLANSSQPWEPLRLHVDKAVSGLRSLTTLLRALGAQEEAISPPDAASAAPLRTVTVDTFRKLFRVYSNFLRGKLMLYTGEACRTGDR

SEQ ID NO: 10 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAFLRGQALLVNSSLPREPLQLHVDKAVSGLRSLTTLLRALGAQEEAISPPDAASAAPLRTITADTFRKLFRVYSNFLRGKLKLYTEEACRTGDR

SEQ ID NO: 11 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLASSSQPWEPLQLHVDKAVSGLRSLTTLLRALGAQEEAISPPDAASAAPLRTITADTFHKLFRVYSNFLRGKLMLYIGEACRTGDR

SEQ ID NO: 12 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLTLLSKAVLRGQALLVNSSQPWEPLRLHVDKAVSGLRSLTTLLRALGAQGEAISPPDAASAAPLRTITVDTFRKLFRVYSNFLRGKLTLYTGEACRTGDR

SEQ ID NO: 13 - protein, 166 aa, Artificial sequence
  Erythropoetin variant
APPRFICDSRVLERYLLEAKEAENIATGCAEHCSLNENIAVPDTKVNFYAWKRMEVGQQAVEVRQGLALLSEAFLRGQALLVNSSPPWEPLQLHVDKAVSGLRSLTALLRALGAQEEAISPPDAASAAPLRTITADTFRKLFRVYSNFLRGKLKLYTGEACRTGDR

SEQ ID NO: 14 - protein, 166 aa, Artificial sequence
  Erythropoetin variant
APPRFICDSRVLERYLLEAKEAENFTTGCAEHCSLNEYITVPDTRVNLYAWKRMEVGQQAVEVRQGLALLSEAVLRGQALLVNSSQPREPLQLHVDKAVSGLRSLTTLLRALGAQEEAISPPDAASATPLRTTTADTFRKLFRVYSNFLRGKLMLYTGEACRTGDR

SEQ ID NO: 15 - protein, 166 aa, Artificial sequence
  Erythropoetin variant
APPRFICDSRVLERYLLEAKEAENFTTGCAEHCSLNENITVPDTKVNFYAWKRMEVGQQAVEVRQGLALLSEAVLRGQALLANSSQPWEPLQLHVDKAVSGLRSLTALLRALGAQEEAISPPDAASAAPLRTATADTFRKLFRVYSNFLRGKLMLYTGEACRTGDR

SEQ ID NO: 16 - protein, 166 aa, Artificial sequence
  Erythropoetin variant
APPRFICDSRVLERYLLEAKEAENFTTGCAEHCSLNENITVPDTKVNFYAWRRMEAGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 17 - protein, 166 aa, Artificial sequence
  Erythropoetin variant
APPRFICDSRVLERYLLEAKEAENITAGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 18 - protein, 166 aa, Artificial sequence
  Erythropoetin variant
APPRFICDSRVLERYILEAKEAENFTMGCAEHCSLNENITVPDTKVNFYAWKRMEVGQQAAEVWQGLALLSEAVLRGQALLVNSSQPWEPLQLHVDKAVSGLRSLTTLLRALGAQEEAISPPDAASAAPLRTITADTFHKLFRVYSNFLRGKLKLYVGEACRTGDR

SEQ ID NO: 19 - protein, 166 aa, Artificial sequence
  Erythropoetin variant
APPRFICVSRVLERYLLEAKEAENIAAGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAAPAAPLRTITADTFRKLFRVYSNFLRGKLKLYTEEACRTGDR

SEQ ID NO: 20 - protein, 166 aa, Artificial sequence
  Erythropoetin variant
APPRFICVSRVLERYLLEAKEAENITAGCAEHCSLNENITVPDTKVNFNAWKRMEVGQQAVEVRQGLALLSEAVLRGQALLANSSQPWGPLQLHVDKAVSGLRSLTTLLRALGAQEEAISPPDAAPAAPLRTITADTFRKLFRVYSNFLRGKLKLYTEEACRTGDR

SEQ ID NO: 21 - protein, 166 aa, Artificial sequence
  Erythropoetin variant
APPRFICDSRVLERYLLEAKEAENIATGCAEHCSLNENITVPDTKVNFYAWKRMEVGQQAVEVRQGLALLSEAVLRGQALLVNSSQPWEPLQLHVDKAVSGLRSLTTLLRALGAQEEAISPPDAASAAPLRTITVDTFRKLFRVYSNFLRGKLKLYTEEACRTGDR

SEQ ID NO: 22 - protein, 166 aa, Artificial sequence
  Erythropoetin variant
APPRFICVSRVLERYLLEAKEAENITTGCAEHCSLNENITVPDTKVNFYAWKRMEVGQQAVEVWQGLALLSEAFLRGQALLVNSSQPWEPLQLHVDKAVSGLRSLTALLRALGAQEEAISPPDAASAAPLRTITADTFRKLFRVYSDFLRGKLKLYTGEACRTGDR

SEQ ID NO: 23 - protein, 166 aa, Artificial sequence
  Erythropoetin variant
APPRFICDSRVLERYLLEAKEAENITSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTEEACRTGDR

SEQ ID NO: 24 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTEEACRTGDR

SEQ ID NO: 25 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTEEACRTGDR

SEQ ID NO: 26 - protein, 166 aa, Artificial sequence
  Erythropoetin variant
APPRFICDSRVLERYLLEAKEAENFTSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTEEACRTGDR

SEQ ID NO: 27 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTEEACRTGDR

SEQ ID NO: 28 - protein, 166 aa, Artificial sequence
  Erythropoetin variant
APPRFICDSRVLERYLLEAKEAENITSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTEEACRTGDR

SEQ ID NO: 29 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTEEACRTGDR

SEQ ID NO: 30 - protein, 166 aa, Artificial sequence
  Erythropoetin variant
APPRFICDSRVLERYLLEAKEAENITTGCAEHCSLNENITVPGTKVNFYAWKRMEVGQQAAEVWQGLALLSEAVRRGQALLANSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 31 - protein, 166 aa, Artificial sequence
  Erythropoetin variant
APPRFICDSRVLERYLLEAKEAENIATGCAEHCSLNENITVPGTKVNFYAWKRMEVGQQAAEVWQGLALLSEAVLRGQALLANSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 32 - protein, 166 aa, Artificial sequence
  Erythropoetin variant
APPRFICDSRVLERYLLEAKEAENITTGCAEHCSLNENITVPGTKVNFYAWKRMEVGQQAAEVWQGLALLSEAVLRGQALLANSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 33 - protein, 166 aa, Artificial sequence
  Erythropoetin variant
APPRFICDSRVLERYLLEAKEAENIATGCAEHCSLNENITVPGTKVNFYAWKRMEVGQQAAEVWQGLALLSEAVRRGQALLVNSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 34 - protein, 166 aa, Artificial sequence
  Erythropoetin variant
APPRFICDSRVLERYLLEAKEAENITTGCAEHCSLNENITVPGTKVNFYAWKRMEVGQQAAEVWQGLALLSEAVRRGQALLVNSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 35 - protein, 166 aa, Artificial sequence
  Erythropoetin variant
APPRFICDSRVLERYLLEAKEAENIATGCAEHCSLNENITVPGTKVNFYAWKRMEVGQQAAEVWQGLALLSEAVLRGQALLVNSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 36 - protein, 166 aa, Artificial sequence
  Erythropoetin variant
APPRFICDSRVLERYLLEAKEAENITTGCAEHCSLNENITVPGTKVNFYAWKRMEVGQQAAEVWQGLALLSEAVLRGQALLVNSSQPWEPLQLHVDKAVSGLRSLTTLLRALGAQEEAISPPDAASAAPLRTTTVDTFRKLFRVYSNFLRGKLKLYIGEACRTGDR

SEQ ID NO: 37 - protein, 166 aa, Artificial sequence
  Erythropoetin variant
APPRFICDSRVLERYLLEAKEAENITAGCAEHCSLNENIAVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 38 - protein, 166 aa, Artificial sequence
  Erythropoetin variant
APPRFICDSRVLERYLLEAKEAEDITAGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 39 - protein, 166 aa, Artificial sequence; identical to SEQ ID NO: 17
  Erythropoetin variant

SEQ ID NO: 40 - protein, 166 aa, Artificial sequence
  Erythropoetin variant
APPRFICDSRVLERYLLEAKEAEDITTGCAEHCSLNENIAVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 41 - protein, 166 aa, Artificial sequence
  Erythropoetin variant
APPRFICDSRVLERYLLEAKEAENITTGCAEHCSLNENIAVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 42 - protein, 166 aa, Artificial sequence
  Erythropoetin variant
APPRFICDSRVLERYLLEAKEAEDITTGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 43 - protein, 166 aa, Artificial sequence
  Erythropoetin variant
APPRFICDSRVLERYLLEAKEAENITTGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 44 - protein, 166 aa, Artificial sequence
  Erythropoetin variant
APPRFTCGSRVLERYLLEAKEAENITTGCAEHCSLNENITVPDTKVNFYAWRRMEVGQQAVEVRQGLALLSEAFLRGQALLVNSSQPWEPLQLHVDKAVSGLRSLTALFRALGAQEEAISPPDAASAAPLRTNTADTFRKLFRVYSNFLRGKLKLYIGEACRTGDR

SEQ ID NO: 45 - protein, 166 aa, Artificial sequence
  Erythropoetin variant
APPRFTCGSRVLERYLLEAREAENITTGCAEHCSLNENITVPDTKVNFYAWKRMEVGQQAVEVRQGLALLSEAFLRGQALLVNSSQPWEPLQLHVDKAVSGLRSLTALFRALGAQEEAISPPDAASAAPLRTNTADTFRKLFRVYSNFLRGKLKLYIGEACRTGDR

SEQ ID NO: 46 - protein, 166 aa, Artificial sequence
  Erythropoetin variant
APPRFTCGSRVLERYLLEAKEAENITTGCAEHCSLNENITVPDTKVNFYAWKRMEVGQQAVEVRQGLALLSEAFLRGQALLVNSSQPWEPLQLHVDKAVSGLRSLTALFRALGAQEEAISPPDAASAAPLRTNTADTFRKLFRVYSNFLRGKLKLYIGEACRTGDR

SEQ ID NO: 47 - protein, 166 aa, Artificial sequence
  Erythropoetin variant
APPRFICDSRVLERYLLEAKEAENITTGVAEHASLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 48 - protein, 166 aa, Artificial sequence
  Erythropoetin variant
APPRFICDSRVLERYLLEAKEAENFTTGCAEHASLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 49 - protein, 166 aa, Artificial sequence
  Erythropoetin variant
APPRFICDSRVLERYLLEAKEAENFTTGVAEHCSLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 50 - protein, 166 aa, Artificial sequence
  Erythropoetin variant
APPRFICDSRVLERYLLEAKEAENITTGCAEHASLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 51 - protein, 166 aa, Artificial sequence
  Erythropoetin variant
APPRFICDSRVLERYLLEAKEAENITTGVAEHCSLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 52 - protein, 166 aa, Artificial sequence
  Erythropoetin variant
APPRFICDSRVLERYLLEAKEAENFTTGCAEHCSLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 53 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLVNSSQPWEPLRLHVDKAVSGLRSLTTLLRALGAQEEAISPPDAASAAPLRTVTADTFRKLFRVYSNFLRGKLKLYTEEACRTGDR

SEQ ID NO: 54 - protein, 166 aa, Artificial sequence
  Erythropoetin variant
APPRFICDSRVLERYLLEAKEAENITAGCAEHCSLNENITVPDTRVDFNAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 55 - protein, 166 aa, Artificial sequence
  Erythropoetin variant
APPRFICDSRVLERYLLEAKEAENIATGCAEHCSLNENITVPDTRVDFNAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 56 - protein, 166 aa, Artificial sequence
  Erythropoetin variant
APPRFICDSRVLERYLLEAKEAENITTGCAEHCSLNENITVPDTRVDFNAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 57 - protein, 166 aa, Artificial sequence
  Erythropoetin variant
APPRFICDSRVLERYLLEAKEAENIAAGCAEHCSLNENITVPDTRVDFYAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 58 - protein, 166 aa, Artificial sequence; identical to SEQ ID NO: 55
  Erythropoetin variant

SEQ ID NO: 59 - protein, 166 aa, Artificial sequence
  Erythropoetin variant
APPRFICDSRVLERYLLEAKEAENITAGCAEHCSLNENITVPDTRVDFYAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 60 - protein, 166 aa, Artificial sequence
  Erythropoetin variant
APPRFICDSRVLERYLLEAKEAENIAAGCAEHCSLNENITVPDTRVDFNAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 61 - protein, 166 aa, Artificial sequence
  Erythropoetin variant
APPRFICDSRVLERYLLEAKEAENIAAGCAEHCSLNENITVPDTRVDFYAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 62 - protein, 166 aa, Artificial sequence
  Erythropoetin variant
APPRFICDSRVLERYLLEAKEAENIATGCAEHCSLNENITVPDTRVDFYAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 63 - protein, 166 aa, Artificial sequence
  Erythropoetin variant
APPRFICDSRVLERYLLEAKEAENITAGCAEHCSLNENITVPDTRVDFYAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 64 - protein, 166 aa, Artificial sequence
  Erythropoetin variant
APPRFICDSRVLERYLLEAKEAENITTGCAEHCSLNENITVPDTRVDFNAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 65 - protein, 166 aa, Artificial sequence
  Erythropoetin variant
APPRFICDSRVLERYLLEAKEAENITTGCAEHCSLNENITVPDTRVDFYAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 66 - protein, 166 aa, Artificial sequence
  Erythropoetin variant
APPRFICDSRVLERYLLEAKEAENITAGCAEHCSLNENITVPDTRVDFNAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 67 - protein, 166 aa, Artificial sequence
  Erythropoetin variant
APPRFICDSRVLERYLLEAKEAENITTGCAEHCSLNENITVPDTRVDFYAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 68 - protein, 166 aa, Artificial sequence
  Erythropoetin variant
APPRFICDSRVLERYLLEAKEAENIATGCAEHCSLNENITVPDTRVDFNAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 69 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLANSSQPWEPLRLHVDKAVSGLRSLTTLLRALGAQEEAISPPDAASAAPLRTVTVDTFRKLFRVYSNFLRGKLMLYTGEACRTGDR

SEQ ID NO: 70 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWEPLRLHVDKAVSGLRSLTTLLRALGAQEEAISPPDAASAAPLRTVTVDTFRKLFRVYSNFLRGKLMLYTGEACRTGDR

SEQ ID NO: 71 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVNSSQPWEPLRLHVDKAVSGLRSLTTLLRALGAQEEAISPPDAASAAPLRTVTVDTFRKLFRVYSNFLRGKLMLYTGEACRTGDR

SEQ ID NO: 72 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAFLRGQALLVNSSQPREPLQLHVDKAVSGLRSLTTLLRALGAQEEAISPPDAASAAPLRTITADTFRKLFRVYSNFLRGKLKLYTEEACRTGDR

SEQ ID NO: 73 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLANSSQPWEPLQLHVDKAVSGLRSLTTLLRALGAQEEAISPPDAASAAPLRTITADTFHKLFRVYSNFLRGKLMLYIGEACRTGDR

SEQ ID NO: 74 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLASSSQPWEPLQLHVDKAVSGLRSLTTLLRALGAQEEAISPPDAASAAPLRTITADTFHKLFRVYSNFLRGKLMLYIGEACRTGDR

SEQ ID NO: 75 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLVSSSQPWEPLQLHVDKAVSGLRSLTTLLRALGAQEEAISPPDAASAAPLRTITADTFHKLFRVYSNFLRGKLMLYIGEACRTGDR

SEQ ID NO: 76 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLASSSQPWEPLQLHVDKAVSGLRSLTTLLRALGAQEEAISPPDAASAAPLRTITADTFRKLFRVYSNFLRGKLMLYIGEACRTGDR

SEQ ID NO: 77 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLVNSSQPWEPLQLHVDKAVSGLRSLTTLLRALGAQEEAISPPDAASAAPLRTITADTFRKLFRVYSNFLRGKLMLYIGEACRTGDR

SEQ ID NO: 78 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLVSSSQPWEPLQLHVDKAVSGLRSLTTLLRALGAQEEAISPPDAASAAPLRTITADTFHKLFRVYSNFLRGKLMLYIGEACRTGDR

SEQ ID NO: 79 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLANSSQPWEPLQLHVDKAVSGLRSLTTLLRALGAQEEAISPPDAASAAPLRTITADTFHKLFRVYSNFLRGKLMLYIGEACRTGDR

SEQ ID NO: 80 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLASSSQPWEPLQLHVDKAVSGLRSLTTLLRALGAQEEAISPPDAASAAPLRTITADTFRKLFRVYSNFLRGKLMLYIGEACRTGDR

SEQ ID NO: 81 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLANSSQPWEPLQLHVDKAVSGLRSLTTLLRALGAQEEAISPPDAASAAPLRTITADTFRKLFRVYSNFLRGKLMLYIGEACRTGDR

SEQ ID NO: 82 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLVNSSQPWEPLQLHVDKAVSGLRSLTTLLRALGAQEEAISPPDAASAAPLRTITADTFHKLFRVYSNFLRGKLMLYIGEACRTGDR

SEQ ID NO: 83 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLVNSSQPWEPLQLHVDKAVSGLRSLTTLLRALGAQEEAISPPDAASAAPLRTITADTFHKLFRVYSNFLRGKLMLYIGEACRTGDR

SEQ ID NO: 84 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLANSSQPWEPLQLHVDKAVSGLRSLTTLLRALGAQEEAISPPDAASAAPLRTITADTFRKLFRVYSNFLRGKLMLYIGEACRTGDR

SEQ ID NO: 85 - protein, 166 aa, Artificial sequence
  Erythropoetin variant
APPRFICDSRVLERYLLEAKEAENITAGCAEHCSLNENITVPDTKVNFYAWKRMEVGQQAVEVRQGLALLSEAVLRGQALLVSSSQPWEPLQLHVDKAVSGLRSLTTLLRALGAQEEAISPPDAASAAPLRTITADTFRKLFRVYSNFLRGKLMLYIGEACRTGDR

SEQ ID NO: 86 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLALLSKAVLRGQALLVNSSQPWEPLRLHVDKAVSGLRSLTTLLRALGAQGEAISPPDAASAAPLRTITVDTFRKLFRVYSNFLRGKLTLYTGEACRTGDR

SEQ ID NO: 87 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLTLLSEAVLRGQALLVNSSQPWEPLRLHVDKAVSGLRSLTTLLRALGAQGEAISPPDAASAAPLRTITVDTFRKLFRVYSNFLRGKLTLYTGEACRTGDR

SEQ ID NO: 88 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLTLLSKAVLRGQALLVNSSQPWEPLRLHVDKAVSGLRSLTTLLRALGAQEEAISPPDAASAAPLRTITVDTFRKLFRVYSNFLRGKLTLYTGEACRTGDR

SEQ ID NO: 89 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLALLSKAVLRGQALLVNSSQPWEPLRLHVDKAVSGLRSLTTLLRALGAQEEAISPPDAASAAPLRTITVDTFRKLFRVYSNFLRGKLTLYTGEACRTGDR

SEQ ID NO: 90 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLTLLSEAVLRGQALLVNSSQPWEPLRLHVDKAVSGLRSLTTLLRALGAQEEAISPPDAASAAPLRTITVDTFRKLFRVYSNFLRGKLTLYTGEACRTGDR

SEQ ID NO: 91 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLALLSEAVLRGQALLVNSSQPWEPLRLHVDKAVSGLRSLTTLLRALGAQEEAISPPDAASAAPLRTITVDTFRKLFRVYSNFLRGKLTLYTGEACRTGDR

SEQ ID NO: 92 - protein, 166 aa, Artificial sequence
  Erythropoetin variant
APPRFICDSRVLERYLLEAKEAENITTGCAEHCSLNENITVPDTKVNFHAWKRMEVGQQAVEVRQGLALLSEAVLRGQALLVNSSQPWEPLRLHVDKAVSGLRSLTTLLRALGAQGEAISPPDAASAAPLRTITVDTFRKLFRVYSNFLRGKLTLYTGEACRTGDR

SEQ ID NO: 93 - protein, 166 aa, Artificial sequence
  Erythropoetin variant
APPRFICDSRVLERYLLEAKEAENITTGCAEHCSLNENIAVPDTKVNFYAWKRMEVGQQAVEVRQGLALLSEAFLRGQALLVNSSPPWEPLQLHVDKAVSGLRSLTALLRALGAQEEAISPPDAASAAPLRTITADTFRKLFRVYSNFLRGKLKLYTGEACRTGDR

SEQ ID NO: 94 - protein, 166 aa, Artificial sequence
  Erythropoetin variant
APPRFICDSRVLERYLLEAKEAENIATGCAEHCSLNENITVPDTKVNFYAWKRMEVGQQAVEVRQGLALLSEAFLRGQALLVNSSPPWEPLQLHVDKAVSGLRSLTALLRALGAQEEAISPPDAASAAPLRTITADTFRKLFRVYSNFLRGKLKLYTGEACRTGDR

SEQ ID NO: 95 - protein, 166 aa, Artificial sequence
  Erythropoetin variant
APPRFICDSRVLERYLLEAKEAENIATGCAEHCSLNENIAVPDTKVNFYAWKRMEVGQQAVEVRQGLALLSEAFLRGQALLVNSSQPWEPLQLHVDKAVSGLRSLTALLRALGAQEEAISPPDAASAAPLRTITADTFRKLFRVYSNFLRGKLKLYTGEACRTGDR

SEQ ID NO: 96 - protein, 166 aa, Artificial sequence
  Erythropoetin variant
APPRFICDSRVLERYLLEAKEAENITTGCAEHCSLNENIAVPDTKVNFYAWKRMEVGQQAVEVRQGLALLSEAFLRGQALLVNSSQPWEPLQLHVDKAVSGLRSLTALLRALGAQEEAISPPDAASAAPLRTITADTFRKLFRVYSNFLRGKLKLYTGEACRTGDR

SEQ ID NO: 97 - protein, 166 aa, Artificial sequence
  Erythropoetin variant
APPRFICDSRVLERYLLEAKEAENIATGCAEHCSLNENITVPDTKVNFYAWKRMEVGQQAVEVRQGLALLSEAFLRGQALLVNSSQPWEPLQLHVDKAVSGLRSLTALLRALGAQEEAISPPDAASAAPLRTITADTFRKLFRVYSNFLRGKLKLYTGEACRTGDR

SEQ ID NO: 98 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAFLRGQALLVNSSQPWEPLQLHVDKAVSGLRSLTALLRALGAQEEAISPPDAASAAPLRTITADTFRKLFRVYSNFLRGKLKLYTGEACRTGDR

SEQ ID NO: 99 - protein, 166 aa, Artificial sequence
  Erythropoetin variant
APPRFICDSRVLERYLLEAKEAENITTGCAEHCSLNEYITVPDTRVNLYAWKRMEVGQQAVEVRQGLALLSEAVLRGQALLVNSSQPREPLQLHVDKAVSGLRSLTTLLRALGAQEEAISPPDAASATPLRTTTADTFRKLFRVYSNFLRGKLMLYTGEACRTGDR

SEQ ID NO: 100 - protein, 166 aa, Artificial sequence
  Erythropoetin variant
APPRFICDSRVLERYLLEAKEAENFTTGCAEHCSLNENITVPDTRVNLYAWKRMEVGQQAVEVRQGLALLSEAVLRGQALLVNSSQPREPLQLHVDKAVSGLRSLTTLLRALGAQEEAISPPDAASATPLRTTTADTFRKLFRVYSNFLRGKLMLYTGEACRTGDR

SEQ ID NO: 101 - protein, 166 aa, Artificial sequence
  Erythropoetin variant
APPRFICDSRVLERYLLEAKEAENFTTGCAEHCSLNEYITVPDTRVNLYAWKRMEVGQQAVEVRQGLALLSEAVLRGQALLVNSSQPREPLQLHVDKAVSGLRSLTTLLRALGAQEEAISPPDAASAAPLRTTTADTFRKLFRVYSNFLRGKLMLYTGEACRTGDR

SEQ ID NO: 102 - protein, 166 aa, Artificial sequence
  Erythropoetin variant
APPRFICDSRVLERYLLEAKEAENITTGCAEHCSLNENITVPDTRVNLYAWKRMEVGQQAVEVRQGLALLSEAVLRGQALLVNSSQPREPLQLHVDKAVSGLRSLTTLLRALGAQEEAISPPDAASATPLRTTTADTFRKLFRVYSNFLRGKLMLYTGEACRTGDR

SEQ ID NO: 103 - protein, 166 aa, Artificial sequence
  Erythropoetin variant
APPRFICDSRVLERYLLEAKEAENITTGCAEHCSLNEYITVPDTRVNLYAWKRMEVGQQAVEVRQGLALLSEAVLRGQALLVNSSQPREPLQLHVDKAVSGLRSLTTLLRALGAQEEAISPPDAASAAPLRTTTADTFRKLFRVYSNFLRGKLMLYTGEACRTGDR

SEQ ID NO: 104 - protein, 166 aa, Artificial sequence
  Erythropoetin variant
APPRFICDSRVLERYLLEAKEAENFTTGCAEHCSLNENITVPDTRVNLYAWKRMEVGQQAVEVRQGLALLSEAVLRGQALLVNSSQPREPLQLHVDKAVSGLRSLTTLLRALGAQEEAISPPDAASAAPLRTTTADTFRKLFRVYSNFLRGKLMLYTGEACRTGDR

SEQ ID NO: 105 - protein, 166 aa, Artificial sequence
  Erythropoetin variant
APPRFICDSRVLERYLLEAKEAENITTGCAEHCSLNENITVPDTRVNLYAWKRMEVGQQAVEVRQGLALLSEAVLRGQALLVNSSQPREPLQLHVDKAVSGLRSLTTLLRALGAQEEAISPPDAASAAPLRTTTADTFRKLFRVYSNFLRGKLMLYTGEACRTGDR

SEQ ID NO: 106 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLANSSQPWEPLQLHVDKAVSGLRSLTALLRALGAQEEAISPPDAASAAPLRTATADTFRKLFRVYSNFLRGKLMLYTGEACRTGDR

SEQ ID NO: 107 - protein, 166 aa, Artificial sequence
  Erythropoetin variant
APPRFICDSRVLERYLLEAKEAENFTTGCAEHCSLNENITVPDTKVNFYAWKRMEVGQQAVEVRQGLALLSEAVLRGQALLVNSSQPWEPLQLHVDKAVSGLRSLTALLRALGAQEEAISPPDAASAAPLRTATADTFRKLFRVYSNFLRGKLMLYTGEACRTGDR

SEQ ID NO: 108 - protein, 166 aa, Artificial sequence
  Erythropoetin variant
APPRFICDSRVLERYLLEAKEAENITTGCAEHCSLNENITVPDTKVNFYAWKRMEVGQQAVEVRQGLALLSEAVLRGQALLVNSSQPWEPLQLHVDKAVSGLRSLTALLRALGAQEEAISPPDAASAAPLRTATADTFRKLFRVYSNFLRGKLMLYTGEACRTGDR

SEQ ID NO: 109 - protein, 166 aa, Artificial sequence
  Erythropoetin variant
APPRFICDSRVLERYLLEAKEAENITTGCAEHCSLNENITVPDTKVNFYAWRRMEAGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 110 - protein, 166 aa, Artificial sequence
  Erythropoetin variant
APPRFICDSRVLERYLLEAKEAENFTTGCAEHCSLNENITVPDTKVNFYAWRRMEAGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 111 - protein, 166 aa, Artificial sequence
  Erythropoetin variant
APPRFICDSRVLERYLLEAKEAENITTGCAEHCSLNENITVPDTKVNFYAWRRMEAGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 112 - protein, 166 aa, Artificial sequence
  Erythropoetin variant
APPRFICDSRVLERYLLEAKEAENFTTGCAEHCSLNENITVPDTKVNFYAWKRMEAGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 113 - protein, 166 aa, Artificial sequence
  Erythropoetin variant
APPRFICDSRVLERYLLEAKEAENITTGCAEHCSLNENITVPDTKVNFYAWKRMEAGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 114 - protein, 166 aa, Artificial sequence
  Erythropoetin variant
APPRFICDSRVLERYLLEAKEAENFTTGCAEHCSLNENITVPDTKVNFYAWKRMEAGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 115 - protein, 166 aa, Artificial sequence
  Erythropoetin variant
APPRFICDSRVLERYLLEAKEAENFTTGCAEHCSLNENITVPDTKVNFYAWRRMEVGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 116 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 117 - protein, 166 aa, Artificial sequence; identical to SEQ ID NO: 110
  Erythropoetin variant

SEQ ID NO: 118 - protein, 166 aa, Artificial sequence
  Erythropoetin variant
APPRFICDSRVLERYLLEAKEAENITTGCAEHCSLNENITVPDTKVNFYAWKRMEAGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 119 - protein, 166 aa, Artificial sequence
  Erythropoetin variant
APPRFICDSRVLERYLLEAKEAENFTTGCAEHCSLNENITVPDTKVNFYAWRRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 120 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 121 - protein, 166 aa, Artificial sequence; identical to SEQ ID NO: 114
  Erythropoetin variant

SEQ ID NO: 122 - protein, 166 aa, Artificial sequence
  Erythropoetin variant
APPRFICDSRVLERYLLEAKEAENITTGCAEHCSLNENITVPDTKVNFYAWRRMEVGQQAVEVWQGLALLSEAVLRGQALLVSSSQPWGPLRLHVDKAVSGLRSLTTLLRALGAQEEAISPPDAASAAPLRTITADTFRKLFRVYSNFLRGKLKLYTEEACRTGDR

SEQ ID NO: 123 - protein, 166 aa, Artificial sequence
  Erythropoetin variant
APPRFICDSRVLERYLLEAKEAENFTTGCAEHCSLNENITVPDTKVNFYAWRRMEAGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 124 - protein, 166 aa, Artificial sequence
  Erythropoetin variant
APPRFICDSRVLERYLLEAKEAENITTGCAEHCSLNENITVPDTKVNFYAWRRMEAGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 125 - protein, 166 aa, Artificial sequence
  Erythropoetin variant
APPRFICDSRVLERYLLEAKEAENFTTGCAEHCSLNENITVPDTKVNFYAWRRMEVGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 126 - protein, 166 aa, Artificial sequence
  Erythropoetin variant
APPRFICDSRVLERYLLEAKEAENITTGCAEHCSLNENITVPDTKVNFYAWRRMEVGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 127 - protein, 166 aa, Artificial sequence; identical to SEQ ID NO: 125
  Erythropoetin variant

SEQ ID NO: 128 - protein, 166 aa, Artificial sequence
  Erythropoetin variant
APPRFICDSRVLERYLLEAKEAENITTGCAEHCSLNENITVPDTKVNFYAWRRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 129 - protein, 166 aa, Artificial sequence; identical to SEQ ID NO: 111
  Erythropoetin variant

SEQ ID NO: 130 - protein, 166 aa, Artificial sequence
  Erythropoetin variant
APPRFICDSRVLERYLLEAKEAENITTGCAEHCSLNENITVPDTKVNFYAWRRMEAGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 131 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 132 - protein, 166 aa, Artificial sequence
  Erythropoetin variant
APPRFICDSRVLERYLLEAKEAENFTTGCAEHCSLNENITVPDTKVNFYAWKRMEAGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 133 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 134 - protein, 166 aa, Artificial sequence
  Erythropoetin variant
APPRFICDSRVLERYLLEAKEAENITTGCAEHCSLNENITVPDTKVNFYAWKRMEAGQQAVEVWQGLALLSEAVLRGQALLVSSSQPWEPLRLHVDKAVSGLRSLTTLLRALGAQEEAISPPDAASAAPLRTITADTFRKLFRVYSNFLRGKLKLYTEEACRTGDR

SEQ ID NO: 135 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVNSSQPWEPLRLHVDKAVSGLRSLTTLLRALGAQEEAISPPDAASAAPLRTITADTFRKLFRVYSNFLRGKLKLYTEEACRTGDR

SEQ ID NO: 136 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWGPLRLHVDKAVSGLRSLTTLLRALGAQEEAISPPDAASAAPLRTITADTFRKLFRVYSNFLRGKLKLYTEEACRTGDR

SEQ ID NO: 137 - protein, 166 aa, Artificial sequence; identical to SEQ ID NO: 43
  Erythropoetin variant

SEQ ID NO: 138 - protein, 166 aa, Artificial sequence
  Erythropoetin variant
APPRFICDSRVLERYLLEAKEAENITAGCAEHCSLNENITVPDTRVNFYAWKRMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 139 - protein, 166 aa, Artificial sequence
  Erythropoetin variant
APPRFICDSRVLERYLLEAKEAENITAGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPLRTITVDTFRKLFRVYSNFLRGKLKLYIEEACRTGDR

SEQ ID NO: 140 - protein, 166 aa, Artificial sequence
  Erythropoetin variant
APPRFICDSRVLERYLLEAKEAENITAGCAEHCSLNENITVPDTRVNFYAWKRMEVGQQAVEVRQGLALLSEAVLRGQALLVNSSQPWEPLQLHVDKAVSGLRSLTTLLRALGAQEEAISPPDAASAAPLRTITVDTFRKLFRVYSNFLRGKLKLYIEEACRTGDR

SEQ ID NO: 141 - protein, 166 aa, Artificial sequence
  Erythropoetin variant
APPRFICDSRVLERYLLEAKEAENITTGCAEHCSLNENITVPDTRVNFYAWERMEVGQQAVEVRQGLALLSEAVLRGQALLVNSSQPWEPLQLHVDKAVSGLRSLTTLLRALGAQEEAISPPDAASAAPLRTITVDTFRKLFRVYSNFLRGKLKLYIEEACRTGDR

SEQ ID NO: 142 - protein, 166 aa, Artificial sequence
  Erythropoetin variant
APPRFICDSRVLERYLLEAKEAENITTGCAEHCSLNENITVPDTRVNFYAWKRMEVGQQAVEVRQGLALLSEAVLRGQALLVNSSQPWEPLQLHVDKAVSGLRSLTTLLRALGAQEEAISPPDAASAAPLRTITVDTFRKLFRVYSNFLRGKLKLYIEEACRTGDR

SEQ ID NO: 143 - protein, 166 aa, Artificial sequence
  Erythropoetin variant
APPRFICDSRVLERYLLEAKEAENITTGCAEHCSLNENITVPDTRVNFYAWKRMEVGQQAVEVRQGLALLSEAVLRGQALLVNSSQPWEPLQLHVDKAVSGLRSLTTLLRALGAQEEAISPPDAASAAPPRTITVDTFRKLFRVYSNFLRGKLKLYIEEACRTGDR

SEQ ID NO: 144 - protein, 166 aa, Artificial sequence; identical to SEQ ID NO: 18
  Erythropoetin variant

SEQ ID NO: 145 - protein, 166 aa, Artificial sequence; identical to SEQ ID NO: 19
  Erythropoetin variant

SEQ ID NO: 146 - protein, 166 aa, Artificial sequence; identical to SEQ ID NO: 20
  Erythropoetin variant

SEQ ID NO: 147 - protein, 166 aa, Artificial sequence; identical to SEQ ID NO: 21
  Erythropoetin variant

SEQ ID NO: 148 - protein, 166 aa, Artificial sequence; identical to SEQ ID NO: 22
  Erythropoetin variant

SEQ ID NO: 149 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTGEACRTGDR

SEQ ID NO: 150 - protein, 166 aa, Artificial sequence
  Erythropoetin variant
APPRFICDSRVLERYLLEAKEAENITSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTGEACRTGDR

SEQ ID NO: 151 - protein, 166 aa, Artificial sequence
  Erythropoetin variant
APPRFICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTGEACRTGDR

SEQ ID NO: 152 - protein, 166 aa, Artificial sequence
  Erythropoetin variant
APPRFICDSRVLERYLLEAKEAENFTSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTGEACRTGDR

SEQ ID NO: 153 - protein, 166 aa, Artificial sequence
  Erythropoetin variant
APPRFICDSRVLERYLLEAKEAENFTTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTGEACRTGDR

SEQ ID NO: 154 - protein, 166 aa, Artificial sequence
  Erythropoetin variant
APPRFICDSRVLERYLLEAKEAENITSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTGEACRTGDR

SEQ ID NO: 155 - protein, 166 aa, Artificial sequence
  Erythropoetin variant
APPRFICDSRVLERYLLEAKEAENFTSGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFHKLFRVYSNFLRGKLKLYTGEACRTGDR

(19) United States
(12) Patent Application Publication
(10) Pub. No.: US 2008/0194475 A1
(43) Pub. Date: Aug. 14, 2008

(54) Erythropoietin Protein Variants

(76) Inventors: Andrew Buchanan, Cambridge (GB); Lutz Jermutus, Cambridge (GB)

Correspondence Address: KLARQUIST SPARKMAN, LLP, 121 SW SALMON STREET, SUITE 1600, PORTLAND, OR, 97204, US

(21) Appl. No.: 11/587,235

(22) PCT Filed: Apr. 22, 2005
(86) PCT No.: PCT/GB2005/001557

§ 371 Date: Jul. 25, 2007

Related U.S. Application Data
(60) Provisional application No. 60/565,231, filed on Apr. 23, 2004.
(60) Provisional application No. 60/580,994, filed on Jun. 17, 2004.
(60) Provisional application No. 60/642,215, filed on Jan. 5, 2005.

(30) Foreign Application Priority Data
Apr. 23, 2004 (GB) 0409122.9

(51) Int. Cl. C07K 14/505; C12N 15/18; C12N 15/63; C12P 21/02; C12Q 1/68; A61K 38/22

(52) U.S. Cl. 514/12; 530/399; 536/23.51; 435/320.1; 435/69.4; 435/6

(57) ABSTRACT

Erythropoietin (EPO) variants, in particular variants that have improved stability, their manufacture and use, for example in therapy.

The present invention relates to erythropoeitin variants, in particular variants that have improved stability. It further relates to manufacture and use of the variants, for example in therapy.

Erythropoietin (EPO) is a member of the hematopoietic growth factor family and behaves as a hormone. It is responsible for the regulation of red blood cell (erythrocyte) production (erythropoiesis), maintaining the body's red blood cell mass at an optimum level. EPO production is stimulated by reduced oxygen content in the renal arterial circulation, mediated by a transcription factor that is oxygen-sensitive. EPO is a produced primarily by cells of the peritubular capillary endothelium of the kidney. Secreted EPO binds to EPO receptors on the surface of bone marrow erythroid precursors, resulting in their rapid replication and maturation to functional red blood cells. This stimulation results in a rapid rise in erythrocyte counts and a consequent rise in hematocrit (% of red blood cells in blood) (D'Andrea et al Cell 1989 57: 277-285. Lodish et al Cold Spring Harb Symp Quant Biol 1995 60: 93-104).

Human EPO was first cloned and amino acid sequence reported by Lin et al. (Proc Natl Acad Sci USA 1985 82: 7582-4) and Jacobs K et al. (Nature 313: 806-810 1985).

Human EPO is an acidic glycoprotein with a molecular weight of approximately 30400 daltons. It is composed of an invariant 165 amino acid single polypeptide chain containing four cysteine residues (at positions 7, 29, 33 and 161), which form the internal disulphide bonds (Lai et al. J Biol Chem 1986 261: 3116-3121; Recny et al J Biol Chem 1987 262: 17156-17163). The disulphide bridge between cysteine 7 and 161 is known to be essential for biological activity. The carbohydrate portion of EPO consists of three N-linked sugars chains at Asn 24, 38 and 83, and one O-linked sugar at Ser 126 (Browne J K et al Cold spring Harb symp Quant Biol 1986 51: 693-702 Egrie J C et al Immunbiology 1986 172: 213-224.)

The structure of human EPO has been reported (Cheetham et al 1988 Nat Struct Biol 5:861-866; Syed et al 1998 Nature 395:511-516). Human EPO is a four helix bundle, typical of members of the hematopoietic growth factor family. In contrast to the invariant amino acid sequence, the carbohydrate structures are variable, a feature referred to as micro-heterogeneity. The differences in carbohydrate moieties, in terms of the branching pattern, complexity size and charge has profound effects on the pharmacokinetics and pharmacodynamics of EPO. The effects of different glycosylation patterns have been well studied (Darling et al 2002 Biochemistry 41: 14524-14531; Storring et al 1998 Br J Haematol 100: 79-89; Halstenson et al 1991 Clin Pharmacol Ther 50: 702-712; Takeuchi et al 1990 J Biol Chem 265: 12127-12130).

The following EPOs have the same amino acid sequence as recombinant human EPO (rhEPO) and variations in the methods of production and glycosylation distinguish these products. Epoetin alfa (genomic DNA) and epoetin beta (cDNA) are described in U.S. Pat. Nos. 4,703,008 and 5,955,422. These have the same amino acid sequence as human EPO and are produced in chinese hamster ovary (CHO) cells. Epoetin alfa is available under the trade names procrit (Ortho Biotech), eprex (Johnson & Johnson), epogin (Chugai) or epogen (Amgen). Epoetin beta is available under the trade name neorecormon or recormon (Hoffmann-La Roche). It was developed by the Genetics Institute for the treatment of anaemia associated with renal disease. Epoetin omega described in U.S. Pat. No. 5,688,679 has the same amino acid sequence as human EPO and is produced in baby hamster kidney cells (BHK-21). Epoetin omega is available under the trade names EPOMAX (Elanex).

Darbepoetin alfa (novel erythropoiesis stimulating protein, NESP) was developed by Amgen and is available under the trade name ARANESP (Macdougall I C, Kidney Int Suppl. 2002 May;(80):55-61). It was designed to contain five N-linked carbohydrate chains (two more than rhEPO). The amino acid sequence of Aranesp differs from that of rhEPO at five substitutions (Ala30Asn, His32Thr, Pro87Val, Trp88Asn, Pro90Thr), thus allowing for additional oligosaccharide attachment at asparagine residues at position 30 and 88. Due to its increased carbohydrate content, Aranesp differs from rhEPO as a result of a higher molecular weight (37,100 compared to 30,400 Daltons), sialic acid content (22 compared to 14 sialic acid residues) and increased negative charge. The increased carbohydrate content of Aranesp accounts for its distinct biochemical and biological properties, in particular a 3-fold longer circulating half-life than other existing erythropoietins when administered via the intravenous (IV) or subcutaneous (SC) route. However, the relative EPO receptor binding affinity was inversely correlated with the carbohydrate content, with Aranesp displaying a 4.3-fold lower relative affinity for the EPO receptor than that of rhEPO. Following SC administration, the absorption of Aranesp is slow and rate-limiting, serum levels reaching a maximum at a mean of 54 h. The time to maximum concentration is longer than that reported for rhEPO, probably because of the increased molecular size of Aranesp. However currently, the extended circulating half-life gives Aranesp a significant clinical advantage over Procrit due to its less frequent dosing. Opportunities may exist however, to explore possible improvements to the affinity of Aranesp for its receptor or to address the rate of absorption following SC administration.

Transkaryotic Therapies (in conjunction with Aventis Pharma) are developing erythropoietin stimulant Dynepo (epoetin delta). Dynepo is a gene-activated human erythropoietin produced in human cell culture, for the treatment of anemia in patients with renal failure.

Roche is developing R-744, continuous erythropoietin receptor activator (CERA), a second-generation erythropoietin, for the potential treatment of anemia associated with chemotherapy. CERA contains a single methoxypolyethylene glycol polmer of approximately 30 Kda that extends the half life of this agent.

Many EPO individual point mutants have been made to study the EPO structure activity relationship (Elliot et al 1997 Blood 89: 493-502; Elliot et al 1996 Blood 87: 2702-2713; Syed et al 1998 Nature 395: 511-516) or effects of glycoslyation (O'Narhi et al 2001 Protein Engineering 14: 135-140; Bill et al 1995 Biochimica et Biophysica Acta 1261: 35-43; Yamaguchi et al 1991 J Biol Chem 266: 20434-20439). There is no significant overlap between those published mutations and those disclosed in the present application. The four mutations where the amino acid mutations do overlap (Lys 20 Arg, Thr 27 Ala, Val 61 Ala and Thr 107 Ala) were previously reported not to affect EPO folding or bioactivity (Elliot et al 1997 Blood 89: 493-502) and the amino acid variations observed at these positions so far do not significantly overlap with the variations disclosed in this invention.

EPO is a major biopharmaceutical product with world-wide sales topping US$ 3 billion. It is used primarily to boost erythrocyte and red blood cell formation in patients to treat anaemia associated with chronic renal failure, cancer chemotherapy, HIV infection, pediatric use, premature infants and to reduce the need for blood transfusions in anaemic patients undergoing elective non-cardiac and non-vascular surgery.

### BRIEF DESCRIPTION OF THE FIGURES

FIG. 1A shows percentage relative abundance (% RA) of EPO WT and EPO variants of SEQ ID NO :17 and SEQ ID NO :9 following HEK EBNA expression and affinity purification, as assessed by SEC-HPLC.

FIG. 1B shows SEC-HPLC trace of affinity purified WT EPO and EPO variant of SEQ ID: 17.

FIG. 2A shows percentage relative abundance (% RA) of wild type EPO stored at 45° C. over two weeks and analysed by SEC-HPLC.

FIG. 2B shows % RA of EPO variant of SEQ ID NO:17 stored at 45° C. over two weeks and analysed by SEC-HPLC.

FIG. 3 shows results of TF1 proliferation assay of wild type EPO and EPO variant of SEQ ID NO: 9 following purification compared to rhEPO:

| | EC50 (pM) |
|---|---|
| EPO WT | 18 |
| rhEPO (RDI) | 27 |
| Variant (SEQ ID: 9) | 4 |

FIG. 4 shows results of TF-1 proliferation assay of wild type EPO and EPO variants after 2 weeks at 5° C. and 45° C.

FIG. 4A shows results for WT EPO.

FIG. 4B shows results for the EPO variant of SEQ ID NO: 9.

FIG. 4C shows results for the EPO variant of SEQ ID NO: 17.

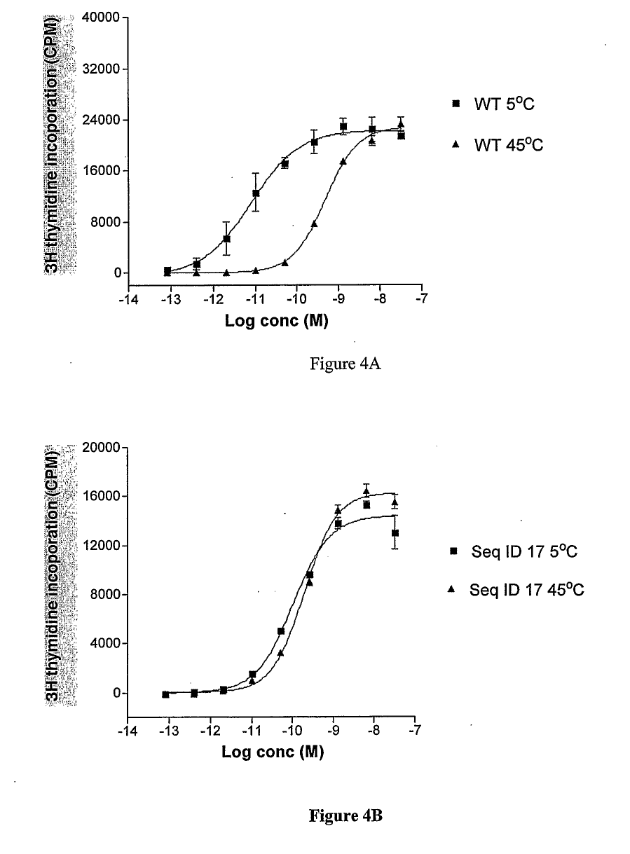

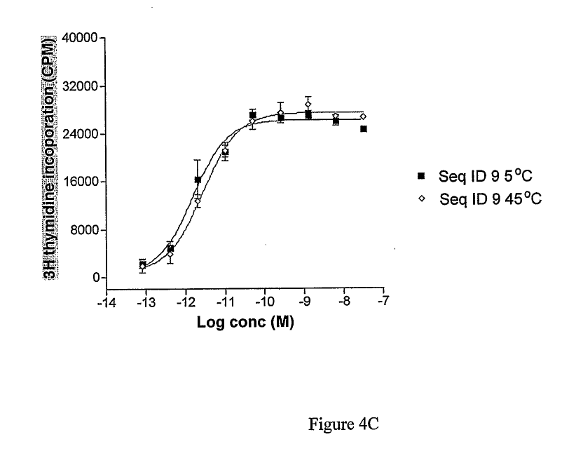

EC50 (pM) of each sample outlined in FIG. 4A, FIG. 4B and FIG. 4C:

| EC50 | Wild type | EPO variant | EPO variant |
|---|---|---|---|
| (pM) | EPO | (SEQ ID NO: 17) | (SEQ ID NO: 9) |
| 5° C. | 8 | 102 | 1.5 |
| 45° C. | 473 | 194 | 2.8 |

The present invention provides EPO variants with improved stability.

Stability can generally be defined as the propensity of the molecule to remain in its folded and active state. Naturally occurring molecules are usually of limited stability as their metabolism, and often their fast metabolism, is a key characteristic of their intrinsic mechanism of action in the body.

Usually, a stable protein in its folded and native structure cannot be degraded by proteases or other mechanisms. It is due to two key off pathways from the stable state by which proteins are usually eliminated in the body. These two are unfolding and aggregation. They are usually linked. Unfolding is the pathway of reverting the folded active molecule into a less folded state. Aggregation is the result of misfolding such that the molecule irreversibly turns into a non-active state. Both unfolding and aggregation significantly increase the protein's susceptibility to proteolytic or other digestion. In the present invention we have modified the folding and unfolding pathway of EPO such that the resulting entity is more stable. While the evolution of increased thermodynamic stability of proteins has been demonstrated previously (Jermutus et al, 2001 Proc Natl Acad Sci 98: 75-80) this is the first description that in vitro evolution and the resulting amino acid changes from this process can result in tangible benefits of bio-therapeutics.

According to one aspect of the present invention there is provided an EPO variant with improved stability compared with wild-type human EPO.

Further aspects and embodiments of the present invention are disclosed herein in and preferred aspects and embodiments are subject to the claims included below.

A measure of stability employed in the context of the present invention can be expressed as a ratio of ability of an EPO variant to bind EPO receptor in the presence of dithiothreitol (DTT), e.g. 10 mM DTT, as determined in a radioimmunoassay (RIA), and ability of the EPO variant to bind the EPO receptor in the absence of DTT in the same radioimmunoassay. The greater the value of the ratio, the greater the stability of the EPO variant and hence its existence in folded state in a reducing environment.

Compared with wild-type EPO, an EPO variant may have such a ratio that is improved at least about five-fold, more preferably at least about ten-fold, fifteen-fold, twenty-fold, twenty-five-fold or thirty-fold.

Variants with improved stability are identified in the experiments described below. See for instance Table 1, which provides measured ratios of EPO receptor binding in the presence and absence of DTT for wild-type EPO (0.02) and various variants (ranging from 0.13 to 0.51).

A further measure of stability employed in the context of the present invention can be expressed as a ratio of ability of an EPO variant to bind EPO receptor in the presence of DTT, e.g. 10 mM DTT, as determined by ELISA using an anti EPO HRP fused antibody, and ability of the EPO variant to bind the EPO receptor in the absence of DTT in the same ELISA. A comparison of the relative binding of an EPO variant to the EPO receptor in the absence and presence of DTT provides an indication of stability. When binding is measured as a percentage value, the greater the percentage value, the greater the stability of the EPO variant and hence its existence in a folded state in a reducing environment.

Compared with wild-type EPO, a preferred EPO variant may have such a percentage value of at least about 10%, 20%, 30% or 40%, more preferably, of at least about 50%, 60% or 70%.

Variants with improved stability are identified in the experiments described below. See for instance Table 3, which provides a percentage value determined by EPO receptor binding in the presence and absence of DTT for wild-type EPO (1%) and various variants (ranging from 1% to 75%).

Another measure of stability that may be employed in the context of the present invention is to compare aggregation of an EPO variant over time with that of wild type EPO. For example, both wild type EPO and variant EPO can be stored at a range of temperatures (for example from 5° C. to 45° C.) and then analysed for breakdown products and aggregated material using routine methods known in the art. A stable protein better remains in folded state and is less prone to breakdown and aggregation.

An EPO variant polypeptide with improved stability may retain 90% residual activity at a temperature that is 2-10 degrees higher at which wild-type protein retains 90% residual activity, e.g. 2, 3, 4, 5, 6, 7, 8, 9 or 10 degrees C. higher. The percentage of residual (i.e. folded, active) protein may be measured by routine biochemical techniques such as HPLC, SDS PAGE or by activity assays such as binding assays or eliciting a response from cells.

Variants with improved stability generally provide for a higher expression and higher yield in downstream processing which results in improved cost of goods (COG). Further, EPO variants with improved stability have an improved shelf life. Longer shelf life is beneficial as it also influences the cost of goods.

An EPO variant with improved stability may have increased efficacy in the body, resulting from a longer half life. Further, an EPO variant with improved stability may be more amenable to routes of administration such as subcutaneous administration, because of reduced aggregation, which not only increases efficacy but also reduces the risk of neutralising or binding antibodies being elicited.

Preferred EPO variants in accordance with the present invention comprise a set of mutations as identified herein.

Further preferred EPO variants in accordance with the present invention have an amino acid sequence selected from SEQ ID NO:'s 3, 4, 5, 6, 7, 8, 9, 10, 11, 12, 13, 14, 15, 16 and 17.

Further preferred EPO variants have SEQ ID NO:'s 18, 19, 20, 21 and 22.

Each and every one of the variants disclosed herein represents an aspect of the invention, as do encoding nucleic acid, a vector comprising such nucleic acid, a host cell comprising such a vector, a composition comprising a variant, a variant of the invention for use in a method of treatment of the human or animal body, use of a variant in the manufacture of a medicament for treatment of anaemia, a method of making the variant and other compositions, methods and uses as disclosed herein.

An EPO variant according to the present invention may contain one or more additional changes compared with the starting protein or with the wild-type or natural protein. A number of different modifications to EPOs are known (both naturally occurring mutants and artificially created variants) with modified properties compared with wild-type. One or more of these properties may be retained or provided in an EPO variant according to the present invention.

Furthermore, an EPO variant according to the present invention containing one or more additional changes compared with the starting protein or with the wild-type or natural protein may show increased stability attributable to the synergistic combination of the one or more mutations.

Wild type EPO has four cysteine residues at positions 7, 29, 33 and 161, which form two intra-molecular disulphide bridges. Preferably the EPO variants of the present invention have an even number of cysteine residues, preferably no more than four cysteine residues, or more preferably no more than two cysteine residues. Preferably EPO variants in accordance with the invention have four cysteine residues or two cysteine residues. It is preferred that the cysteine residues are at positions 7, 29, 33 and 161, more preferably at positions 7 and 161.

The presence of an odd number of cysteine residues is undesirable as this may result in aggregation due to cross-linking of molecules by inter-molecular disulphide bridges. In a variant containing an odd number of cysteine residues, the unpaired cysteine should be removed and replaced with either the original wild type residue if not originally cysteine or with any amino acid residue for example, alanine.

If the cysteine residue at either position 29 or position 33 is mutated, it is preferred that the unpaired cysteine residue is paired by reverting the mutation at position 29 or position 33 back to its original cysteine residue.

If the unpaired cysteine residue is at position 29, one option is to replace this unpaired cysteine with any other amino acid. A further option is to replace the unpaired cysteine at position 29 with valine and further replace the amino acid residue at position 33 with alanine. Valine and alanine are known to have a similar steric requirement as a disulphide bridge so are considered as useful alternatives for a disulphide bridge (Worn and Pluckthun, 1998, FEBS Letter 427:357-361).

Further, if position 29 of the EPO variant has an amino acid residue other than cysteine, it is preferable to have either tyrosine or arginine at position 33.

It is known in the art that increased carbohydrate content of EPO variants increases the circulating half-life (there are five N-linked glycosylation sites, two more than wild type EPO, in ARANESP). It is preferred that an EPO variant of the present invention, especially when expressed in a mammalian expression system, has no less than five, preferably no less than four, more preferably no less than three N-linked glycosylation sites. Preferably a variant of the invention has 5, 4 or 3 N-linked glycosylation sites. The number of N-linked glycosylation sites is irrelevant if the EPO variant is expressed in a prokaryotic expression system. For mammalian expression of an EPO variant of the present invention, the number of N-linked glycosylation sites is preferably restored to the preferred number.

Preferred EPO variants in accordance with the present invention have an amino acid sequence selected from SEQ ID NOs: 3, 6, 7, 9 and 17. A more preferred variant of the present invention has the amino acid sequence set out in SEQ ID NO: 17.

Further preferred variants include those in which one or more mutations are made in addition to any of the sets of mutations identified herein, and those in which one or more cysteine residues or one or more residues involved in N-glycosylation (i.e. N or Z wherein N is asparagine and Z is serine or threonine occurring in a motif NXZ, and wherein X is any amino acid except proline. Thus, for example, for a variant for which a set of mutations is disclosed herein in which there is a change at a cysteine or an asparagine or other residue involved in N-glycosylation, further variants of this are provided by the present invention in which there is a reversion to cysteine (in accordance with the principles set out elsewhere herein) and/or a reversion to provide an N-glycosylation motif. Furthermore, where in a set of mutations disclosed herein there is no change at a cysteine or an asparagine or other residue involved in N-glycosylation, further variants are provided by the present invention in which there are provided one or more mutations at a cysteine (in accordance with the principles set out elsewhere herein) and/or one or more mutations to provide one or more additional sites for N-glycosylation.

Embodiments of further variants provided by the present invention include any with an addition mutation at any one or more of the following positions: 6, 29, 33, 45, 47, 48, 49, 61, 64, 74, 88, 92, 107, 109, 133, 135, 154, 157 and 158. The residues provided at any one or more of these positions may be selected from those identified in the following table:

| Position | Amino acid residue |
|---|---|
| 6 | T |
| 29 | V, A |
| 33 | A, R, Y |
| 45 | R |
| 47 | D |
| 48 | L |
| 49 | Y, N |
| 61 | A |
| 64 | R |
| 74 | F |
| 88 | R |
| 92 | R |
| 107 | A |
| 109 | F |
| 133 | T, N, V, A |
| 135 | V |
| 154 | T, M |
| 157 | I |
| 158 | E |

Preferred variants according to the invention are also set out here, in conjunction with identification of the initial set of mutations on which they are based:

Initial set of mutations (found in SEQ ID NO: 3) I25F T27S R139H G158E

Further Variants:

T27S R139H G158E;

I25F R139H G158E;

R139H G158E;

I25F T27S G158E;

I25F G158E;

T27S G158E;

G158E.

Initial set of mutations (found in SEQ ID NO: 4) T26A D43G V61A L75R V82A I133T A135V T157I

Further Variants:

D43G V61A L75R V82A I133T A135V T157I;

T26A D43G V61A V82A I133T A135V T157I;

D43G V61A V82A I133T A135V T157I;

T26A D43G V61A L75R I133T A135V T157I;

T26A D43G V61A L75R V82A I133T A135V T157I;

D43G V61A L75R I133T A135V T157I;

T26A D43G V61A I133T A135V T157I;

D43G V61A I133T A135V T157I;

Initial set of mutations (found in SEQ ID NO: 5) N24D T27A T40A K45R K52E W64R L130P A135V T157I G158E

Further Variants:

T27A T40A K45R K52E W64R L130P A135V T157I G158E;

N24D T27A K45R K52E W64R L130P A135V T157I G158E;

T27A K45R K52E W64R L130P A135V T157I G158E;

N24D T40A K45R K52E W64R L130P A135V T157I G158E;

T40A K45R K52E W64R L130P A135V T157I G158E;

N24D K45R K52E W64R L130P A135V T157I G158E;

K45R K52E W64R L130P A135V T157I G158E.

Initial set of mutations (found in SEQ ID NO: 6) I6T D8G K20R K52R W64R V74F T107A L109F I133N T157I

Further Variants:

I6T D8G K52R W64R V74F T107A L109F I133N T157I;

I6T D8G K20R W64R V74F T107A L109F I133N T157I;

I6T D8G W64R V74F T107A L109F I133N T157I;

Initial set of mutations (found in SEQ ID NO: 7)

I25F C29V C33A W64R Q92R I133V G158E

Further Variants:

C29V C33A W64R Q92R I133V G158E;

I25F C33A W64R Q92R I133V G158E;

I25F C29V W64R Q92R I133V G158E;

C33A W64R Q92R I133V G158E;

C29V W64R Q92R I133V G158E;

I25F W64R Q92R I133V G158E;

W64R Q92R I133V G158E.

Initial set of mutations (found in SEQ ID NO: 8)

T26A T27A K45R N47D Y49N E89G Q92R G158E

Further Variants:

T27A K45R N47D Y49N E89G Q92R G158E;

T26A K45R N47D Y49N E89G Q92R G158E;

K45R N47D Y49N E89G Q92R G158E;

T26A T27A K45R N47D E89G Q92R G158E;

T26A K45R N47D E89G Q92R G158E;

T27A K45R N47D E89G Q92R G158E;

T26A T27A K45R N47D Y49N Q92R G158E;

T26A T27A K45R N47D Q92R G158E.

T26A K45R N47D Q92R G158E;

T27A K45R N47D Q92R G158E;

K45R N47D Y49N Q92R G158E;

K45R N47D Q92R G158E;

T27A K45R N47D Y49N Q92R G158E;

K45R N47D E89G Q92R G158E; T26A K45R N47D Y49N Q92R G158E.

Initial set of mutations (found in SEQ ID NO: 9)

I25F V82A Q92R I133V A135V K154M

Further Variants:

V82A Q92R I133V A135V K154M;

I25F Q92R I133V A135V K154M;

Q92R I133V A135V K154M.

Initial set of mutations (found in SEQ ID NO: 10)

V74F Q86L W88R G158E

Further Variants:

V74F W88R G158E.

Initial set of mutations (found in SEQ ID NO: 11)

T27A W64R V82A N83S R139H K154M T157I

Further Variants:

T27A W64R V82A R139H K154M T157I;

W64R V82A N83S R139H K154M T157I;

T27A W64R N83S R139H K154M T157I;

T27A W64R V82A N83S K154M T157I;

W64R K154M T157I;

W64R N83S R139H K154M T157I;

W64R V82A R139H K154M T157I;

W64R V82A N83S K154M T157I;

W64R V82A K154M T157I;

W64R R139H K154M T157I;

T27A W64R R139H K154M T157I;

T27A W64R V82A K154M T157I;

T27A W64R N83S K154M T157I.

Initial set of mutations (found in SEQ ID NO: 12)

Y49H W64R A68T E72K Q92R E116G A135V K154T

Further Variants:

Y49H W64R E72K Q92R E116G A135V K154T;

Y49H W64R A68T Q92R E116G A135V K154T;

Y49H W64R A68T E72K Q92R A135V K154T;

Y49H W64R E72K Q92R A135V K154T;

Y49H W64R A68T Q92R A135V K154T;

Y49H W64R Q92R A135V K154T;

Y49H W64R Q92R E116G A135V K154T.

Initial set of mutations (found in SEQ ID NO: 13) T26A T40A W64R V74F Q86P T107A

Further Variants:

T40A W64R V74F Q86P T107A;

T26A W64R V74F Q86P T107A;

T26A T40A W64R V74F T107A;

T40A W64R V74F T107A;

T26A W64R V74F T107A;

W64R V74F T107A;

Initial set of mutations (found in SEQ ID NO: 14)

I25F N38Y K45R F48L W64R W88R A128T I133T K154M

Further Variants:

N38Y K45R F48L W64R W88R A128T I133T K154M;

I25F K45R F48L W64R W88R A128T I133T K154M;

I25F N38Y K45R F48L W64R W88R I133T K154M;

K45R F48L W64R W88R A128T I133T K154M;

N38Y K45R F48L W64R W88R I133T K154M;

I25F K45R F48L W64R W88R I133T K154M;

K45R F48L W64R W88R I133T K154M.

Initial set of mutations (found in SEQ ID NO: 15) I25F W64R V82A T107A I133A K154M

Further Variants:

W64R V82A T107A I133A K154M;

I25F W64R T107A I133A K154M;

W64R T107A I133A K154M.

Initial set of mutations (found in SEQ ID NO: 16) I25F K52R V56A N83S E89G Q92R G158E

Further Variants:

K52R V56A N83S E89G Q92R G158E;

I25F K52R V56A E89G Q92R G158E;

K52R V56A E89G Q92R G158E;

I25F V56A N83S E89G Q92R G158E;

V56A N83S E89G Q92R G158E;

I25F V56A E89G Q92R G158E;

I25F K52R N83S E89G Q92R G158E;

I25F N83S E89G Q92R G158E;

I25F K52R V56A E89G Q92R G158E;

V56A E89G Q92R G158E;

I25F K52R E89G Q92R G158E;

N83S E89G Q92R G158E;

I25F V56A E89G Q92R G158E;

K52R N83S E89G Q92R G158E;

I25F K52R V56A N83S Q92R G158E;

K52R V56A N83S Q92R G158E;

I25F K52R N83S Q92R G158E;

K52R N83S Q92R G158E;

I25F K52R N83S Q92R G158E;

K52R Q92R G158E;

K52R V56A E89G Q92R G158E;

K52R V56A Q92R G158E;

N83S E89G Q92R G158E;

N83S Q92R G158E;

I25F V56A N83S Q92R G158E;

I25F N83S Q92R G158E;

V56A N83S Q92R G158E;

Q92R G158E;

V56A N83S Q92R G158E;

I25F E89G Q92R G158E.

Initial set of mutations (found in SEQ ID NO: 17)

T27A K45R K52E W64R L130P A135V T157I G158E

Further Variants:

K45R K52E W64R L130P A135V T157I G158E;

T27A K45R W64R L130P A135V T157I G158E;

T27A K45R K52E W64R A135V T157I G158E;

T27A K45R W64R A135VT 157I G158E;

K45R K52E W64R A135V T157I G158E;

K45R W64R A135V T157I G158E;

K45R W64R L130P A135V T157I G158E.

Further variants have been identified using a further selection method, with mutations as follows

L16I I25F T27M V61A R139H T157V

D8V T26A T27A S126P G158E

D8V T27A Y49N W64R V82A E89G I26P G158E

T26A W64R A135V G158E

D8V V74F T107A N147D.

Each of the sets of mutations disclosed herein may be included within an EPO variant that has a set of mutations consisting of the identified sets of mutations. Each of these sets of mutations may be included within an EPO variant comprising the identified set of mutations and one or more additional mutations, especially one or more mutations disclosed herein as preferred mutations.

A polypeptide according to the present invention may be isolated and/or purified (e.g. using an antibody) for instance after production by expression from encoding nucleic acid (for which see below). Thus, a polypeptide may be provided free or substantially free from contaminants. A polypeptide may be provided free or substantially free of other polypeptides. The isolated and/or purified polypeptide may be used in formulation of a composition, which may include at least one additional component, for example a pharmaceutical composition including a pharmaceutically acceptable excipient, vehicle or carrier. A composition including a polypeptide according to the invention may be used in prophylactic and/or therapeutic treatment as discussed below.

A convenient way of producing a polypeptide according to the present invention is to express nucleic acid encoding it, by use of the nucleic acid in an expression system. Accordingly, the present invention also encompasses a method of making a polypeptide (as disclosed), the method including expression from nucleic acid encoding the polypeptide (generally nucleic acid according to the invention). This may conveniently be achieved by growing a host cell in culture, containing such a vector, under appropriate conditions which cause or allow expression of the polypeptide. Polypeptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. 'phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Nucleic acid encoding a polypeptide of the invention is provided as a further aspect of the invention.

Generally, nucleic acid according to the present invention is provided as an isolate, in isolated and/or purified form, or free or substantially free of contaminants. Nucleic acid may be wholly or partially synthetic and may include genomic DNA, cDNA or RNA.

Nucleic acid may be provided as part of a replicable vector, and also provided by the present invention are a vector including nucleic acid encoding an EPO variant of the invention, particularly any expression vector from which the encoded polypeptide can be expressed under appropriate conditions, and a host cell containing any such vector or nucleic acid. An expression vector in this context is a nucleic acid molecule including nucleic acid encoding a polypeptide of interest and appropriate regulatory sequences for expression of the polypeptide, in an in vitro expression system, e.g. reticulocyte lysate, or in vivo, e.g. in eukaryotic cells such as COS or CHO cells or in prokaryotic cells such as E. coli.

A further aspect of the present invention provides a host cell containing nucleic acid as disclosed herein. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

A still further aspect provides a method which includes introducing the nucleic acid into a host cell. The introduction, which may (particularly for in vitro introduction) be generally referred to without limitation as “transformation” or “transfection”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

Following production of an EPO variant by expression, its activity, for example its ability to bind EPO receptor, or ability to elicit cell proliferation can be tested routinely.

According to a further aspect of the present invention there is provided a method of making an EPO variant with improved stability over wild-type human EPO, the method comprising:
producing the EPO variant by expression from encoding nucleic acid;
testing the EPO variant for improved stability compared with wild-type human EPO, for example using as a measure of stability ratio of binding activity to EPO receptor in the presence of DTT in a radioimmunoassay and of binding activity to EPO receptor in the absence of DTT in the same assay.

The ratio may be improved at least five-fold or more as indicated elsewhere herein.

The variant may be a variant containing a set of mutations as disclosed herein with one or more additional mutations and/or one or more reversions to wild-type, e.g. to restore or introduce or remove a cysteine residue, to restore or introduce or remove a N-glycosylation site or to restore or introduce an O-linked glycosylation site. Conservative substitution is also preferred in various embodiments of the present invention, e.g. at one or more positions identified elsewhere herein.

By “conservative substitution” is meant substitution of a first amino acid residue with a second, different amino acid residue, wherein the first and second amino acid residues have side chains which have similar biophysical characteristics. Similar biophyical characteristics include hydrophobicity, charge, polarity, capability of providing or accepting hydrogen bonds. Examples of conservative substitutions include changing serine to threonine or tryptophan, glutamine to asparagine, lysine to arginine, alanine to valine, aspartate to glutamate, valine to isoleucine, asparagine to serine.

Such a method may optionally include isolating and/or purifying the EPO variant following its production and prior to testing.

Someone performing the method may additionally perform a prior step of providing an EPO variant by altering the amino acid sequence of the EPO variant, e.g. by substitution and/or insertion of one or more amino acids as discussed. Various different variants may be provided and tested for the desired activity, e.g. in order to identify from a range of variants one or more variants with the properties desired in accordance with the present invention. Normally, alteration of the amino acid sequence of EPO will be made by altering the coding sequence of nucleic acid encoding EPO. One or more nucleotides may be altered to alter one or more codons and thus the encoded amino acid(s). As mentioned elsewhere herein, and will be apparent to those skilled in the art, any suitable technique for mutagenesis, especially directed or site-specific mutagenesis, can be employed in order to change the coding sequence, and thus the encoded amino acid sequence, for an EPO variant.

A further aspect of the present invention provides a method of identifying or obtaining an EPO variant which has improved stability compared with wild-type human EPO, the method comprising:
mutating nucleic acid encoding an EPO polypeptide to provide one or more nucleic acids with sequences encoding one or more EPO polypeptides with altered amino acid sequences (“EPO variants”);
expressing the nucleic acid or nucleic acids to produce the encoded EPO variant or variants;
testing the EPO variant or variants thus produced for improved stability compared with wild-type human EPO.

A library or diverse population of EPO variants may be produced and tested for the desired abilities.

Mutation may be at any residue identified within a set of mutations as disclosed herein, any cysteine and/or any residue at which N-glycosylation occurs (e.g. in wild-type sequence N24, N38 or N83) or a residue that contributes to recognition of an arginine for N-glysosylation (e.g. in wild-type sequence residue 26 or 40).

The EPO polypeptide that is subject to the mutation may comprise any set of mutations disclosed herein and may have an amino acid sequence selected from SEQ ID NO: 3, 4, 5, 6, 7, 8, 9, 10, 11, 12, 13, 14, 15, 16 and 17.

Selection from a library or diverse population may employ a display system such as phage display and/or ribosome display (for review, see Lowe D and Jermutus L, 2004, Curr. Pharm, Biotech, 517-27; WO92/01047). Selection for variants of improved stability may involve comparison of binding or other indicator of activity when the variants are produced in the presence and absence of DTT, e.g. as disclosed herein.

One or more EPO variants with the desired properties may be identified or selected.

After an EPO variant of the invention has been identified or obtained it may be provided in-isolated and/or purified form, it may be used as desired, and it may be formulated into a composition comprising at least one additional component, such as a pharmaceutically acceptable excipient or carrier. Nucleic acid encoding the EPO variant may be used to produce the variant for subsequent use. As noted, such nucleic acid may, for example, be isolated from a library or diverse population initially provided and from which the EPO variant was produced and identified.

An EPO variant in accordance with the present invention may be used in methods of diagnosis or treatment of the human or animal body of subjects, preferably human.

Accordingly, further aspects of the invention provide methods of treatment comprising administration of an EPO variant as provided, pharmaceutical compositions comprising such an EPO variant, and use of such an EPO variant in the manufacture of a medicament for administration, for example in a method of making a medicament or pharmaceutical composition comprising formulating the EPO variant with a pharmaceutically acceptable excipient.

Clinical indications in which an EPO variant may be used to provide therapeutic benefit include anaemia, for example anaemia associated with chronic renal failure, cancer chemotherapy or HIV infection, or paediatric use, in premature infants, or to reduce the need for blood transfusions in anaemic patients undergoing elective non-cardiac and non-vascular surgery.

In accordance with the present invention an EPO variant may be given to an individual, preferably by administration in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be any suitable route, but most likely injection (with or without a needle), especially subcutaneous injection. Other preferred routes of administration include administration by inhalation or intranasal administration.

For intravenous, subcutaneous or intramuscular injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Further aspects and embodiments of the present invention will be apparent to those skilled in the art in the light of the present disclosure, including the following experimental exemplification.

All documents mentioned anywhere in this specification are incorporated by reference.

### Experimental

### EXAMPLE 1

EPO cDNA was obtained from Invitrogen, and libraries of variants created and subjected to rounds of selection and further mutation by error prone PCR with an error rate of 8.1 nucleotide mutations/molecule. This introduced 4 mutations per molecule and a library of approximately 2.5×10¹⁰ variant molecules.

Particular variants were isolated and characterised as described below.

Selections for Stability

In vitro translations and selections were performed in the presence and absence of DTT as described in Jermutus et al 2001, with the following exceptions:

1. Translations were carried out at 30° C. for 10 minutes.

2. EPO wild type (WT) was included to demonstrate improvement over wild type.

3. The number of PCR cycles was determined with real time PCR. Incorporating real time PCR allows the user to select the number of PCR cycles to amplify specific product. This minimises non-specific background and allows comparison of the output improvement over wild type by calculating relative quantitation values (RQV).

In brief, selections were performed whereby, following incubation with the library with EPO receptor fusion protein, the fusion protein was captured and the bound tertiary complexes (mRNA-ribosome-EPO variant) were recovered by magnetic separation whilst unbound complexes were washed away. The mRNA encoding the bound EPO variants were then recovered by RT-PCR and the selection process repeated with increasing concentrations of DTT (0.5 mM increasing to 10 mM over 4 rounds).

Round 1 selection was performed at 0.5 mM DTT and the PCR product from this was progressed to a round 2 selection at 10 mM DTT. The PCR product from round two selections was of identical strength to the wild type control (RQV=1). Following round 2, two selection strands were then followed.

In the first strand (3A) the PCR product from round 2 was progressed through two more rounds of selection in 10 mM DTT. The round 4A PCR product showed enrichment for more stable variants as shown on a gel and by RQV (Round 3A RQV=6, round 4A RQV=11).

For the second strand (3B) the round 2 PCR product was further mutagenised using error prone PCR with an error rate of 4 mutations per molecule to further increase the library size. Following mutagenesis the library of mutants were selected in round 3B at 5 mM DTT and at round 4 B at 10 mM DTT. As shown on the gel and the RQV, round 4B showed enrichment for more stable variants over wild type and variants isolated from round 4A (Round 4B RQV=23).

The original library prior to selection and the PCR products of round 4A and round 4B outputs were cloned into the in vitro expression vector pIVEX2.3d (Roche). In brief the outputs were PCR amplified to introduce a 5′ Nco1 restriction site and at the 3′ end a stop codon followed immediately by a Not1 restriction site. The stop codon allowed the expression of untagged variant EPO. The product was gel purified, double digested with Not1 and Nco1 (New England Biolabs) and gel purified. The digested product was ligated into Not1 Nco1 digested pIVEX2.3d and transformed into E. coli TG1 cells. Individual colonies were picked into 96 well plates for screening and sequencing.

Screening of Single EPO Variants in Primary Stability RIA

EPO variants were screened for stability using the primary stability RIA (radio immunoassay) as described in Jermutus et al 2001. In brief for each variant a linear DNA template was amplified, transcribed, the mRNA purified on G25 sephadex columns and quantified. For each variant in vitro translations in the presence of ³⁵S-labelled methionine were set up in duplicate at 30° C. for 30 minutes, one in non-reducing conditions and one in 10 mM DTT (dithiothreitol). The translations were stopped with PBS with 0.05% Tween 20, with DTT at the same concentration as the translations. The translation mixture was incubated on a plate coated with EPO receptor for 1 hour at room temperature. Plates were washed three times in PBS with 0.05% Tween 20 and three times in PBS. The remaining radioactivity was eluted with 0.1 M triethylamine and quantified by liquid scintillation counting. A measure of the variants' stability was calculated by dividing the RIA signal in 10 mM DTT by the RIA signal in the absence of DTT. The more stable the variant the bigger the ratio i.e. if inactive in DTT the ratio=0, if fully active in DTT the ratio=1.

Eighty-five cloned EPO variants from a round of selection were screened as described above. From this 20 EPO variants were identified that had ratios greater than WT. These variants were retested and from these 16 EPO variants were identified that were more stable than WT (Table 1). These EPO variants were shown to be specific for the cognate receptor by demonstrating that they did not bind growth hormone receptor or BSA.

Sequence Analysis of EPO Variants

The EPO variants were sequenced, the sequence of the 16 more stable variants is described below as how they differ at the amino acid level from WT EPO SEQ ID NO: 2.

SEQ ID NO: 3

I25F T27S R139H G158E

SEQ ID NO: 4

T26A D43G V61A L75R V82A I133T A135V T157I

SEQ ID NO: 5

N24D T27A T40A K45R K52E W64R L130P A135V T157I G158E

SEQ ID NO: 6

I6T D8G K20R K52R W64R V74F T107A L109F I133N T157I

SEQ ID NO: 7

I25F C29V, C33A, W64R Q92R I133V G158E

SEQ ID NO: 8

T26A T27A K45R N47D Y49N E89G Q92R G158E

SEQ ID NO: 9

I25F V82A Q92R I133V A135V K154M

SEQ ID NO: 10

V74F Q86L W88R G158E

SEQ ID NO: 11

T27A W64R V82A N83S R139H K154M T157I

SEQ ID NO: 12

Y49H W64R A68T E72K Q92R E116G A135V K154T

SEQ ID NO: 13

T26A T40A W64R V74F Q86P T107A

SEQ ID NO: 14

I25F N38Y K45R F48L W64R W88R A128T I133T K154M

SEQ ID NO: 15

I25F W64R V82A T107A I133A K154M

SEQ ID NO: 16

I25F K52R V56A N83S E89G Q92R G158E

SEQ ID NO: 17

T27A K45R K52E W64R L130P A135V T157I G158E

Secondary Stability Assay

In this assay, EPO variants are expressed with and without tags in a mammalian cell line e.g. CHO or HEK, purified and tested for biological activity.

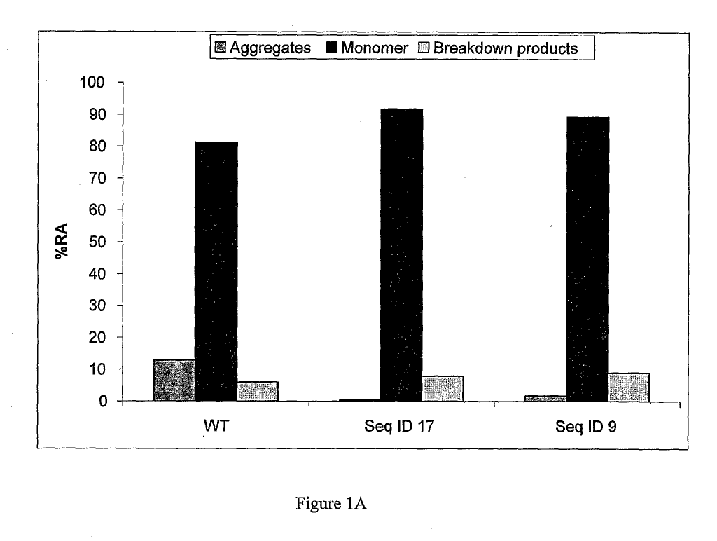

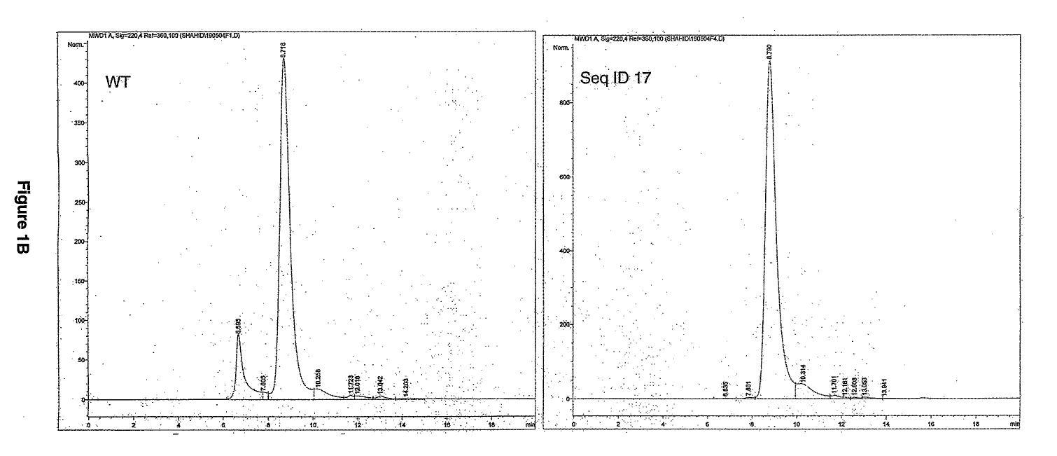

The EPO variants showed a reduction in the proportion of aggregates following expression and purification as assessed by SEC-HPLC (FIG. 1).

The stability of these purified variants is assessed in a secondary stability screen in comparison to the EPO WT they were derived from and commercial forms.

Protein Aggregation is Measured Over Time.

In brief, protein samples were stored in sterile glass vials with metal crimped lids at 5° C., 37° C. and 45° C. for two weeks. At set time points (week zero, week one and week two), samples were analysed by HPLC, SDS-PAGE reducing and non-reducing, absorbance and IEF to identify differences in aggregation and breakdown products between selected variants and WT.

The activity of the WT and variants is also measured in a relevant cell assay for example a TF-1 cell proliferation assay (see Example 2 below).

Results

Here, aggregates were referred to as high molecular weight species and breakdown products as low molecular weight species relative to the EPO monomer. Wild type EPO and representative EPO variants of the present invention were stable at 5° C. with no change in proportions of monomer, aggregates and breakdown products, as assessed by SEC-HPLC and SDS-PAGE.

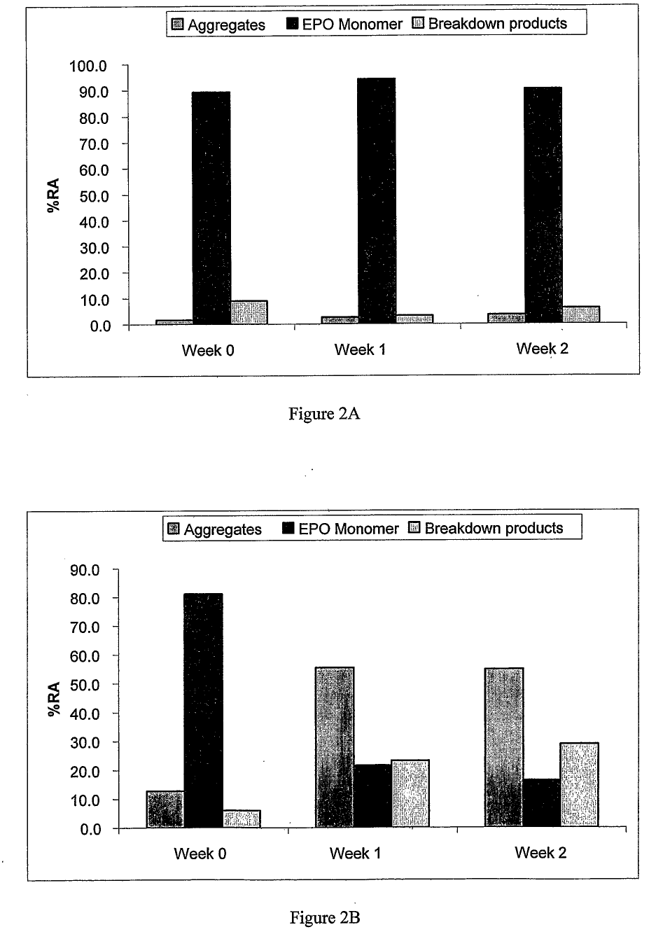

However, wild type EPO was unstable at 37° C. and 45° C. as detected by SEC-HPLC and SDS-PAGE (reducing and non reducing). SEC-HPLC revealed that the proportion of monomer reduced from 80% at time nought to 20% with a concomitant rise in aggregates and breakdown products (FIG. 2A) for wild type EPO samples at 45° C. at week 2.

The EPO variants, (SEQ ID NO: 17 and SEQ ID NO: 9), appeared more stable in this assay. There was no significant change in the proportion of monomer, aggregates and breakdown products over the two weeks at 37° C. and 45° C., as assessed by SDS-PAGE and SEC-HPLC. This is illustrated with data for the EPO variant of SEQ ID NO: 17 in FIG. 2B.

### EXAMPLE 2

### Potency of EPO Variants in the TF-1 Cell Proliferation Assay

The potency of wild type EPO and EPO variants of the present invention were assessed using a TF-1 cell proliferation assay.

TF-1 are a human premyeloid cell line established from a patient with erythroleukemia (Kitamura et al 1989, Blood:73 p 375-80). The TF-1 cell line is factor dependent for survival and proliferation. In this respect TF-1 cells responded to EPO and were maintained in media containing human GM-CSF (4 ng/ml, R&D Systems). EPO dependent proliferation was determined by measuring the reduction in incorporation of tritiated thymidine into the newly synthesized DNA of dividing cells.

TF-1 Assay Protocol

TF-1 cells were obtained from R&D Systems and maintained according to supplied protocols. Assay media comprised RPMI-1640 with GLUTAMAX I containing 5% foetal bovine serum and 1% sodium pyruvate. Prior to each assay, TF-1 cells were pelleted by centrifugation at 300×g for 5 mins, the media removed by aspiration and the cells resuspended in assay media. This process was repeated three additional times with cells resuspended at a final concentration of 10⁵ /ml in assay media. EPO variants (in triplicate) were diluted to the desired concentrations in assay media. 100 μl of resuspended cells were then added to each assay point to give a total assay volume of 200 μl/well. Assay plates were incubated at 37° C. was for 72 hours at 37° C. under 5% CO2. 20 μl of tritiated thymidine (5 μCi/ml, NEN) was then added to each assay point and assay plates were returned to the incubator for a further 4 hours. Cells were harvested on glass fibre filter plates (Perkin Elmer) using a cell harvester. Thymidine incorporation was determined using a Packard TopCount microplate liquid scintillation counter. Data were analysed using Graphpad Prism software.

Results

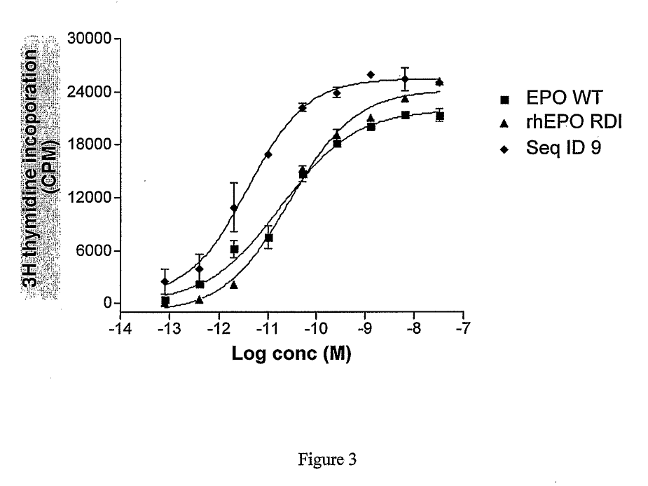

Wild type EPO and EPO variants were tested for biological activity in the TF-1 proliferation assay and compared to recombinant human (rh EPO) produced in CHO cells (Research Diagnostics). Wild type EPO had gan EC50 of 18 pM, similar to the EC50 of 27 pM of rhEPO. The representative EPO Variant of SEQ ID NO: 9 had an EC50 of 4 pM, approximately 4.5 fold better than WT EPO (FIG. 3).

The biological activity of the EPO WT and variants from the stability screen were assessed in the TF1 assay. Following two weeks at 5° C. the EC50 of WT EPO and EPO variants was unaffected. However, after a two week incubation at 45° C., the EC50 of wild type EPO was 60 fold higher than the 5° C. In comparison the EPO variants showed only a two fold increase in EC50 (FIG. 4). This demonstrates that the EPO variants are not only more stable than wild type EPO but also more biologically active after a two week incubation at 45° C.

Expression of Wild Ttype EPO and EPO Variants in Mammalian Cell Lines

A cDNA for wild-type human EPO was obtained from Invitrogen. The complete coding region of wild type EPO or EPO variants of the present invention were amplified by PCR and inserted into pEE12.4 expression vector. Stable cell lines of CHO-K1SV cells expressing wild type or variant EPO was established by transfection and subsequent selection for MSX resistance. Purification of the recombinant proteins were then performed on the conditioned media. Initially the sample volume was reduced using 30,000 molecular weight ultrafiltration device (Vivascience). Concentrated sample was diluted 1:1 with 100% ethanol and precipitated material removed by centrifugation, and the remaining solution conditioned by diluting 1:1 with 50 mM MES pH4.75. Ion exchange was then performed using SP sepharose and the recombinant proteins eluted with NaCl. Gel filtration was carried out to obtain monomeric protein using Superdex 75.

Wild type EPO and EPO variants were then purified and formulated in the same commercial formulation of EPOGEN. Samples were tested for stability and biological activity as described in the present examples.

### EXAMPLE 3

### Construction of Library of EPO Variants and Selection of EPO Variants for Improved Stability

Library Construction.

EPO cDNA was obtained from Invitrogen. The mature sequence was reformatted into the ribosome display linear template which was subsequently used for library creation. At the DNA level, a T7 promoter was added at the 5′-end for efficient transcription to mRNA. At the mRNA level, the construct contained a prokaryotic ribosome-binding site (Shine-Dalgarno sequence). At the 3′ end, a portion of gIII was added to act as a spacer (Hanes et al., (2000) Meth Enzymol 328:404). A library of variants was created using error prone PCR, following the manufacturer's protocol (BD Bioscience), with an error rate of 8.1 nucleotide mutations/molecule. This introduced 4 mutations per molecule and a library of approximately 2.5×10¹⁰ variant molecules.

Selections for Stability

Selections were performed with at least two simultaneous stability selection pressures including; DTT, HIC and increased temperature, followed by selection for functional activity.

The reducing agent dithiothreitol (DTT) was present during the translation and selection. DTT prevents disulphide bridge formation, which is an important component of EPO stability. Following translation, the translation mix with DTT was incubated with hydrophobic interaction chromatography (HIC) matrix at 25° C. The combination of DTT, HIC and increased temperature of 25° C. compared with the usual 4° C. should capture and remove the less stable variants that have unfolded and or mis-folded due to the selection pressure. HIC matrix is removed from the mix for example by centrifugation or filtration. A buffer exchange step may also be required before progressing to the functional selection.

In vitro translations and selections were performed in the presence and absence of DTT as described in Jermutus et al., (2001), with the following exceptions:

1. Translations were carried out at 30° C. for 10 minutes.

2. HIC and increased temperature were used as added selection pressures. Following translation with and without DTT the translation was stopped in buffer containing KCl (1 M increasing to 3 M) and DTT at the same concentration as the translation. The translation mix was then incubated with 1 ml bed volume of HIC beads (butyl-, octyl- and phenyl-sepharose, Amersham). After shaking for 30 minutes at 25° C. the HIC beads were removed by centrifugation at room temperature.

3. The supernatant was then cooled to 4° C. and functional selections were performed whereby, following incubation of the stability selected library with EPO receptor fusion protein, the fusion protein was captured and the bound complexes were recovered by magnetic separation whilst unbound complexes were washed away. The mRNA encoding the bound EPO variants was then recovered by RT-PCR and the selection process was repeated. Four rounds of selection were performed with more destabilising combinations of DTT, HIC and increased temperature.

The PCR products from round 4 were cloned into the in vitro expression vector pIVEX2.3d (Roche). In brief the outputs were PCR amplified to introduce a 5′ Nco1 restriction site and at the 3′ end a stop codon followed immediately by a Not1 restriction site. The stop codon allowed the expression of untagged variant EPO. The product was gel purified, double digested with Not1 and Nco1 (New England Biolabs) and gel purified. The digested product was ligated into Not1 Nco1 digested pIVEX2.3d and transformed into E. coli TG1 cells. Individual colonies were picked into 96 well plates for screening and sequencing.

Screening of Single EPO Variants in Primary Stability RIA

EPO variants were screened for stability using the primary stability RIA (radio immunoassay) as described in Jermutus et al., (2001). In brief for each variant a linear DNA template was amplified, transcribed, the mRNA purified on G25 sephadex columns and quantified. For each variant in vitro translations in the presence of ³⁵S-labelled methionine were set up in duplicate at 30° C. for 30 minutes, one in non-reducing conditions and one in 10 mM DTT (dithiothreitol). The translations were stopped with PBS with 0.05% Tween 20, with DTT at the same concentration as the translations. The translation mixture was incubated on a plate coated with EPO receptor for 1 hour at room temperature. Plates were washed three times in PBS with 0.05% Tween 20 and three times in PBS. The remaining radioactivity was eluted with 0.1 M triethylamine and quantified by liquid scintillation counting. A measure of the variants' stability was calculated by dividing the RIA signal in 10 mM DTT by the RIA signal in the absence of DTT. The more stable the variant the bigger the ratio i.e. if inactive in DTT the ratio=0, if fully active in DTT the ratio=1.

Forty eight cloned EPO variants from round 4 were screened as described above. From this 5 EPO variants were identified that were more stable than WT (Table 2).

Sequence Analysis of EPO Variants

The EPO variants from round 4 were sequenced, the sequence of the 5 more stable variants is described below as differences at the amino acid level from WT EPO having the amino acid sequence shown as SEQ ID NO: 2.

SEQ ID NO: 18

L16I I25F T27M V61A R139H T157V

SEQ ID NO: 19

D8V T26A T27A S126P G158E

SEQ ID NO: 20

D8V T27A Y49N W64R V82A E89G S126P G158E

SEQ ID NO: 21

T26A W64R A135V G158E

SEQ ID NO: 22

D8V V74F T107A N147D

### EXAMPLE 4

### Determination of Single and Combinations of Mutations Conferring Improved Stability

To determine mutations which confer improved stability to an EPO variant, 14 combinations of single, double and triple mutations were constructed, with reference to SEQ ID NO: 3 (I25F, T27S, R139H, G158E). The single and combination mutations selected from I25F, T27S, R139H and G158E are as set out in Table 3.

EPO Stability ELISA

EPO variants were translated cell free in the presence and absence of DTT and the plates coated as for the stability RIA. Plates were washed three times in 1×PBS and 50 μl of translated EPO sample were added to each well and incubated for one hour at room temperature. Plates were washed three times in PBS/Tween and 50 μl of anti-EPO HRP conjugate (part 890127 R&D Quantikine kit DEP00) added and incubated for two hours at room temperature. Plates were washed three times in 1×PBS/Tween. 50 μl of TMB was added and the reaction stopped in 0.5M H₂SO₄ when colour developed. The absorbance was read at 450 nm and the relative stability calculated as for the stability RIA.

As shown in Table 3, an increase in the number of mutations increased the stability of the EPO variant, with the improved stability of the variant SEQ ID NO: 3 resulting from the synergistic combination of all four mutations.

TABLE 1
Results of stability RIA for 16 variants with absolute
RIA signal after non-reducing conditions (DTT−) and
measure of stability calculated as described above
(DTT+/DTT−).

| | SEQ ID NO: | DTT− | DTT+/DTT− |
|---|---|---|---|
| WT EPO | SEQ ID NO: 2 | 13879 | 0.02 |
| Variants | SEQ ID NO: 3 | 20107 | 0.36 |
| | SEQ ID NO: 4 | 30012 | 0.40 |
| | SEQ ID NO: 5 | 10199 | 0.39 |
| | SEQ ID NO: 6 | 25445 | 0.37 |
| | SEQ ID NO: 7 | 10807 | 0.38 |
| | SEQ ID NO: 8 | 28556 | 0.30 |
| | SEQ ID NO: 9 | 44726 | 0.26 |
| | SEQ ID NO: 10 | 25311 | 0.26 |
| | SEQ ID NO: 11 | 25680 | 0.25 |
| | SEQ ID NO: 12 | 24603 | 0.22 |
| | SEQ ID NO: 13 | 18118 | 0.20 |
| | SEQ ID NO: 14 | 22625 | 0.16 |
| | SEQ ID NO: 15 | 16382 | 0.15 |
| | SEQ ID NO: 16 | 39249 | 0.13 |
| | SEQ ID NO: 17 | 30973 | 0.51 |

TABLE 2
Results of stability RIA for 5 variants with absolute RIA
signal after non-reducing conditions (DTT−) and measure
of stability calculated as described above (DTT+/DTT−).

| Clone | SEQ ID NO: | DTT− | DTT+/DTT− |
|---|---|---|---|
| WT EPO | SEQ ID NO: 2 | 5009 | 0.00 |
| Variant | | | |
| Var 1 | SEQ ID NO: 18 | 67552 | 0.33 |
| Var 2 | SEQ ID NO: 19 | 4890 | 0.14 |
| Var 3 | SEQ ID NO: 20 | 13553 | 0.12 |
| Var 4 | SEQ ID NO: 21 | 40087 | 0.1 |
| Var 5 | SEQ ID NO: 22 | 14895 | 0.1 |

TABLE 3
Results of stability ELISA for 14 combinations of
mutations based on SEQ ID NO: 3 (combinations of any of
I25F, T27S, R139H and G158E) and measure of stability
calculated as described above (DTT+/DTT−).

| | | ELISA % |
|---|---|---|
| | Point Mutation(s) | DTT+/DTT− |
| WT EPO (SEQ ID NO: 2) | — | 1 |
| Variants | 25 | 6 |
| | 27 | 1 |
| | 139 | 2 |
| | 158 | 3 |
| | 25 + 27 | 6 |
| | 25 + 139 | 7 |
| | 25 + 158 | 11 |
| | 27 + 139 | 2 |
| | 27 + 158 | 3 |
| | 139 + 158 | 7 |
| | 25 + 27 + 139 | 19 |
| | 25 + 27 + 158 | 24 |
| | 25 + 139 + 158 | 33 |
| | 27 + 139 + 158 | 11 |
| SEQ ID NO: 3 | 25 + 27 + 139 + 158 | 75 |

SEQ ID NO: 1

nucleotide sequence encoding wild-type human EPO

```
SEQ ID NO: 2
amino acid sequence of wild-type human EPO
GCCCCACCACGCTTCATCTGTGACAGCCGAGTCCTGGAGAGGTACCTCTTGGAGGCCAAG
  A P P R F I C D S R V L E R Y L L E A K
                                                        10 20
GAGGCCGAGAATATCACGACGGGCTGTGCTGAACACTGCAGCTTGAATGAGAATATCACT
  E A E N I T T G C A E H C S L N E N I T
                                                        30 40
GTCCCAGACACCAAAGTTAATTTCTATGCCTGGAAGAGGATGGAGGTCGGGCAGCAGGCC
  V P D T K V N F Y A W K R M E V G Q Q A
                                                        50 60
GTAGAAGTCTGGCAGGGCCTGGCCCTGCTGTCGGAAGCTGTCCTGCGGGGCCAGGCCCTG
  V E V W Q G L A L L S E A V L R G Q A L
                                                        70 80
TTGGTCAACTCTTCCCAGCCGTGGGAGCCCCTGCAGCTGCATGTGGATAAAGCCGTCAGT
  L V N S S Q P W E P L Q L H V D K A V S
                                                        90 100
GGCCTTCGCAGCCTCACCACTCTGCTTCGGGCTCTGGGAGCCCAGGAGGAAGCCATCTCC
  G L R S L T T L L R A L G A Q E E A I S
                                                        110 120
CCTCCAGATGCGGCCTCAGCTGCTCCACTCCGAACAATCACTGCTGACACTTTCCGCAAA
  P P D A A S A A P L R T I T A D T F R K
                                                        130 140
CTCTTCCCAGTCTACTCCAATTTCCTCCGGGGAAAGCTGAAGCTGTACACAGGGGAGGCC
  L F R V Y S N F L R G K L K L Y T G E A
                                                        150 160
TGCAGGACAGGGGACAGA
  C R T G D R
```

1. An erythropoietin (EPO) variant polypeptide which has at least five-fold improvement in a measure of stability compared with human wild-type EPO of sequence SEQ ID NO: 2, wherein the measure of stability is ratio of binding activity to EPO receptor in the presence of DTT in a radioimmunoassay and of binding activity to EPO receptor in the absence of DTT in the same assay.

2. The EPO variant polypeptide according to claim 1 comprising a set of mutations in the human wild-type sequence of SEQ ID NO: 2 selected from the group consisting of the following sets of mutations:
(1) T27A K45R K52E W64R L130P A135V T157I G158E;
(2) I25F T27S R139H G158E;
(3) T26A D43G V61A L75R V82A I133T A135V T157I;
(4) N24D T27A T40A K45R K52E W64R L130P A135V T157I G158E;
(5) I6T D8G K20R K52R W64R V74F T107A L109F I133N T157I;
(6) I25F C29V C33A W64R Q92R I133V G158E;
(7) T26A T27A K45R N47D Y49N E89G Q92R G158E;
(8) I25F V82A Q92R I133V A135V K154M;
(9) V74F Q86L W88R G158E;
(10) T27A W64R V82A N83S R139H K154M T157I;
(11) Y49H W64R A68T E72K Q92R E116G A135V K154T;
(12) T26A T40A W64R V74F Q86P T107A;
(13) I25F N38Y K45R F48L W64R W88R A128T I133T K154M;
(14) I25F W64R V82A T107A I133A K154M; and
(15) I25F K52R V56A N83S E89G Q92R G158E.

3. The EPO variant polypeptide according to claim 2 which has an amino acid sequence selected from the group consisting of SEQ ID NO's: 17, 3, 4, 5, 6, 7, 8, 9, 10, 11, 12, 13, 14, 15 and 16.

4. The EPO variant polypeptide according to claim 2 comprising one or more additional mutations.

5. The EPO variant polypeptide according to claim 4 comprising a mutation to provide an amino acid at position 29 that is other than cysteine.

6. The EPO variant polypeptide according to claim 4 comprising tyrosine or arginine at position 33.

7. The EPO variant polypeptide according to claim 4 comprising a mutation at one or more positions selected from the group consisting of positions 6, 29, 33, 45, 47, 48, 49, 61, 64, 74, 88, 92, 107, 109, 133, 135, 154, 157 and 158.

8. The EPO variant polypeptide according to claim 7 wherein the residue provided at mutation at said one or more positions is selected from the residues identified in the following table:

| Position | Amino Acid Residue |
|---|---|
| 6 | T |
| 29 | V, A |
| 33 | A, R, Y |
| 45 | R |
| 47 | D |
| 48 | L |
| 49 | Y, N |
| 61 | A |
| 64 | R |
| 74 | F |
| 88 | R |
| 92 | R |
| 107 | A |
| 109 | F |
| 133 | T, N, V, A |
| 135 | V |
| 154 | T, M |
| 157 | I |
| 158 | E |

9. An EPO variant polypeptide which has improved stability compared with wild-type human EPO and comprising a set of mutations in the human wild-type sequence of SEQ ID NO: 2 selected from the group consisting of the following sets of mutations
(1) T27AK45RK52E W64R L130P A135VT157I G158E;
(2) I25F T27S R139H G158E;
(3) T26A D43G V61A L75R V82A I133T A135V T157I;
(4) N24D T27A T40A K45R K52E W64R L130P A135V T157I G158E;
(5) I6T D8G K20R K52R W64R V74F T107A L109F I133N T157I;
(6) I25F C29V C33A W64R Q92R I133V G158E;
(7) T26A T27A K45R N47D Y49N E89G Q92R G158E;
(8) I25F V82A Q92R I133V A135V K154M;
(9) V74F Q86L W88R G158E;
(10) T27A W64R V82A N83S R139H K154M T157I;
(11) Y49H W64R A68T E72K Q92R E116G A135V K154T;
(12) T26A T40A W64R V74F Q86P T107A;
(13) I25F N38Y K45R F48L W64R W88R A128T I133T K154M;
(14) I25F W64R V82A T107A I133A K154M; and
(15) I25F K52R V56A N83S E89G Q92R G158E;
wherein the residue at a position of mutation within the set of mutations is reverted to the residue at that position in wild-type EPO or subject to a conservative amino acid substitution, wherein the position is selected from the group consisting of one or more of residues 20, 24, 25, 26, 27, 29, 33, 38, 40, 49, 52, 56, 68, 72, 75, 82, 83, 86, 89, 116, 128, 130 and 139.

10. The EPO variant polypeptide according to claim 9 wherein the position reverted or subject to a conservative amino acid substitution is selected from the group consisting of one or more of positions 24, 26, 29, 33, 38, 40 and 83.

11. A nucleic acid encoding the EPO variant polypeptide according claim 1.

12. A vector comprising a nucleic acid according to claim 11.

13. A host cell comprising a vector according to claim 12.

14. A composition comprising the EPO variant polypeptide according to claim 1.

15. The composition according to claim 14 comprising a pharmaceutically acceptable excipient.

16-18. (canceled)

19. A method of making an EPO variant polypeptide that has improved stability compared with wild-type human EPO, the method comprising:
producing an EPO variant according to claim 1 by expression from encoding nucleic acid; and
testing for improved stability.

20. The method according to claim 19 comprising the step of isolating the EPO variant prior to the testing.

21. The method according to claim 19 comprising mutating nucleic acid encoding an EPO polypeptide, which is wild-type human EPO polypeptide or an EPO variant polypeptide, to provide a nucleic acid encoding an EPO variant prior to expression therefrom.

22. A method of identifying or obtaining an EPO variant which has improved stability compared with wild-type human EPO, the method comprising:
mutating nucleic acid encoding an EPO variant polypeptide according to claim 1, to provide one or more nucleic acids with sequences encoding one or more EPO polypeptides with altered amino acid sequences (“EPO variants”);
expressing the nucleic acid or nucleic acids to produce the encoded EPO variant or variants; and
testing the EPO variant or variants thus produced for improved stability compared with wild-type human EPO.

23. The method according to claim 22 comprising producing a library of EPO variants and testing the variants of said library for improved stability.

24. The method according to claim 23 comprising identifying one or more EPO variants with improved stability.

25. The method according to claim 24 comprising isolating said one or more EPO variants.

26. The method according to claim 24 comprising isolating nucleic acid sequence encoding said one or more EPO variants.

27. The method according to claim 26 comprising formulating said one or more isolated EPO variants into a composition comprising at least one additional component.

28. A method of treatment comprising administering to an individual in need thereof an EPO variant polypeptide according to claim 1.

29. A nucleic acid encoding the EPO variant polypeptide according claim 9.

30. A composition comprising the EPO variant polypeptide according to claim 9.

31. A method of making an EPO variant polypeptide that has improved stability compared with wild-type human EPO, the method comprising:
producing an EPO variant according to claim 9 by expression from encoding nucleic acid; and
testing for improved stability.

32. A method of identifying or obtaining an EPO variant which has improved stability compared with wild-type human EPO, the method comprising:
mutating nucleic acid encoding an EPO variant polypeptide according to claim 9, to provide one or more nucleic acids with sequences encoding one or more EPO polypeptides with altered amino acid sequences (“EPO variants”);
expressing the nucleic acid or nucleic acids to produce the encoded EPO variant or variants; and
testing the EPO variant or variants thus produced for improved stability compared with wild-type human EPO.

33. A method of treatment comprising administering to an individual in need thereof an EPO variant polypeptide according to claim 9.
